(19) United States
(12) Patent Application Publication
(10) Pub. No.: US 2009/0092637 A1
(43) Pub. Date: Apr. 9, 2009

(54) Medicaments and methods to treat autoimmune disease and cancer

(75) Inventors: Johnny Ludvigsson, Linkoping (SE); Rosaura Casas, Linkoping (SE); Maria Faresjo, Linkoping (SE); Anders Essen-Moller, Stockholm (SE)

Correspondence Address: COHEN & GRIGSBY, P.C., 625 LIBERTY AVENUE, PITTSBURGH, PA, 15222-3152, US

(73) Assignee: Diamyd Therapeutics AB, Stockholm (SE)

(21) Appl. No.: 12/150,115

(22) Filed: Apr. 24, 2008

Related U.S. Application Data
(60) Provisional application No. 60/926,121, filed on Apr. 24, 2007.
(60) Provisional application No. 60/993,640, filed on Sep. 13, 2007.

(51) Int. Cl. A61K 39/00; A61K 39/12; A61P 37/00

(52) U.S. Cl. 424/204.1; 424/184.1; 424/277.1; 424/275.1

(57) ABSTRACT

The present invention relates to methods and formulations for GAD-vaccination to evoke a systemic effect rather that a GAD-specific effect. The present invention may therefore be used in the treatment of disease in humans not bearing on a GAD-specific effect. The invention includes a method to treat an autoimmune disease or disorder by administering at least one sequestered autoantigen in a prime and boost regimen for sensitization purposes followed by a boost for treatment purposes. This may be done upon diagnosis. Examples of sequestered autoantigens include: GAD65, GAD67, Pro-Insulin, Basic Myelin Protein, MOG and Chondrotoin II.

The present invention relates to medications and methods for treatment of autoimmune disease within six months from diagnosis.

### CROSS-REFERENCE TO RELATED APPLICATIONS

This application claims the priority benefit of U.S. Provisional Application Ser. No. 60/926,121, filed Apr. 24, 2007, and U.S. Provisional Application Ser. No. 60/993,640, filed Sep. 13, 2007, which are hereby incorporated in their entirety by reference.

### BACKGROUND OF THE INVENTION

Autoimmune disease, allergy and cancer are diseases which are associated with dysregulated immune inflammation in both similar and different ways. Some autoimmune diseases and tissue rejection mechanisms are organ-specific, mostly T cell-mediated and triggered by environmental factors including viral infections in susceptible individuals. This may lead to exposure of sequestered autoantigens or transplantation antigens, which might be utilized therapeutically to regulate the disease. Allergy is often associated with a B cell-mediated IgE response to repeated allergen exposure. Cancer may be triggered by inflammatory or other factors that alter cell behaviour, but once transformed cancer cells often escape surveillance from the immune system.

In organ-specific autoimmune disease it is possible to ameliorate the disorder by suppressing the immune system or removing certain reactive parts thereof. Immunosuppression may be accomplished using general suppressive agents such as cyclosporine, while use of T cell-specific antibodies (e.g. anti-CD3 antibodies) and B cell-specific antibodies (e.g. anti-CD20-antibodies) represent more modern approaches to silence specifric arms of the immune system.

Immunosuppression and/or depletion/silencing of immunocomponents may interfere with acquired B and T cell memory and in addition result in frequent adverse effects, including reactivation of Epstein Barr infections, influenza and other microorganisms. Moreover, antibodies to major arms of the immune system may, at least temporarily, compromise the treated subjects' capacity to combat emerging cancerous disease. Immunomodulatory therapies using antibodies are cumbersome as they often require hospitalization and i.v infusions for several days.

There is thus a tremendous unmet need to find medicaments and methods for the safe treatment of disorders in which the immune system itself sometimes aggravates and/or causes the disease.

Biological markers such as antibodies to certain autoantigens are often a hallmark of organ-specific autoimmune diseases. Such antibodies are often of the IgG type but certain isotypes are more prominent as disease debut is neared (Jacob Pedersen). Although it is known that the cellular and humoral arms of the immune system act in concert in fighting disease, the current opinion is that in organ-specific autoimmune disease the T-cell response is the primary instigator while the presence of at least some autoantibody isotype classes is a indication that that a cell-mediated response is active against the said autoantigen.

Since autoantigens by definition are endogenous it may be surprising that they are not tolerated by the immune system in autoimmune disease states. Insulin, which is abundant during the development of a subject's immune system, is for example considered to be an autoantigen in type 1 diabetes in which insulin antibodies frequently occur. It is possible that autoreactivity to insulin is induced by environmental, including inflammatory, factors in pancreas-related areas or by normally sequestered pre-forms of insulin. Systemic concentrations of prepro-insulin or pro-insulin for example may not have been presented to the immune system during its development and once exposed an aggressive response to these previously sequestered endogenous autoantigens may result. The response may include cytotoxic T cells that react toward beta cell surface autoantigen epitopes presented by MHC class I and/or class II antigens, or B cells that secrete antibodies to the previously tolerated or sequestered autoantigens. B cells and autoreactive cytotoxic cells may in addition work in unison and maintain the targeted combat.

As insulin is an abundant molecule and well exposed to the immune system its potential as an immunomodulatory autoantigen in type 1 diabetes is weak. It is, however, possible to enhance the immunomodulatory effect of weak antigens by altering them or peptides derived thereof, by coupling them to other stronger antigens, formulating them in adjuvants or exposing them to the immune system via new routes of administration, such as in the case of insulin for example, via the alimentary tract.

Convincing data from the spontaneously diabetic NOD mouse have indicated that GAD65 therapy prevents autoimmune diabetes through induction of functional immune tolerance. A dose-finding study in patients diagnosed with latent autoimmune diabetes in the adult (LADA) within 5 years has demonstrated that 4 doses of 500 μg of recombinant human GAD65 in a standard vaccine formulation can be safely administered to patients and that a prime-and-boost injection of 20 μg and 100 μg doses are able to preserve endogenous residual insulin secretion for several years, while 4 μg and 500 μg doses did not show efficacy. It was therefore a surprising finding, which is the subject of the present invention, that GAD65 treatment in patients with type 1 diabetes does only works in patient groups diagnosed within a six month period prior to first administration and that additional administrations are necessary to maintain the immunomodulatory effect of this sequestered but still endogenous autoantigen.

Type 1 diabetes (T1D) is an autoimmune disease¹ which affects 0.3-1% of the population and its incidence is increasing. Modern intensive insulin therapy has reduced but cannot completely prevent nerve, kidney, eye, and cardiac complications in those with T1D. The disorder has significant morbidity and mortality with many patients experiencing acute and sometimes life threatening complications.²⁻⁴ Even modest residual insulin secretion, with stimulated C-peptide above 0.2 pmol/ml, has been purported to provide clinically meaningful benefits in terms of reducing long-term complications.⁵ However, several approaches to preserve residual beta cell function have been attempted with minimal or too weak effect in relation to the adverse effects⁶⁻¹⁵ except for treatment with anti-CD3 monoclonal antibodies which seems quite promising, although many patients get adverse events.^{16,17} Administration of auto-antigens seems relevant to try.¹⁸ Insulin and glutamic acid decarboxylase 65 (GAD65) are major autoantigens in T1D^{19,20} and have been tested in immunomodulation experiments.²¹ Earlier data from the spontaneously diabetic NOD mouse have indicated that GAD65 prevents T1D by induction of functional immune tolerance.^{22,23} Previously, a dose-finding study in patients with latent autoimmune diabetes in the adult (LADA), indicated that a prime and boost injection of 20 μg Diamyd® (recombinant human GAD65 in a standard vaccine formulation with alum) may provide preserved residual insulin secretion.²⁴

In view of these and other findings, we designed and initiated investigations addressing whether administration of Diamyd® in young T1D patients of recent onset was safe and could reduce or halt the loss of residual insulin secretion. Here we report the results after a 15-month study period.

### SUMMARY OF THE INVENTION

The present invention relates to methods and formulations for GAD-vaccination to evoke a systemic effect rather that a GAD-specific effect. The present invention may therefore be used in the treatment of disease in humans not bearing on a GAD-specific effect.

In general terms, the invention includes a method to treat an autoimmune disease or disorder by administering at least one sequestered autoantigen in a prime and boost regimen for sensitization purposes followed by a boost for treatment purposes. This may be done upon diagnosis. It is preferred that the sequestered autoantigen is at least one selected from the group consisting of: GAD65, GAD67, Pro-Insulin, Basic Myelin Protein, MOG and Chondrotoin II.

The administration may be through one or more of oral, nasal, inhaled, intramuscular, or subcutaneous administration(s).

Included in the diseases or disorders to which the present invention may be directed are those selected from the group consisting of pancreatitis, pseudomembranous colitis, acute ulcerative colitis, chronic ulcerative colitis, achalasia, cholangitis, Crohn's disease, inflammatory bowel disease, enteritis, Whipple's disease, diabetes, asthma, allergy, immune complex disease, organ ischemia, organ necrosis, hay fever, eosinophilic granuloma, granulomatosis, sarcoidosis, vaginitis, prostatitis, urethritis, bronchitis, emphysema, rhinitis, cystic fibrosis, burns, dermatitis, dermatomyositis, urticaria, vasulitis, cardiovascular disease, atherosclerosis, pericarditis, myocarditis, myocardial ischemia, periarteritis nodosa, rheumatic fever, rheumatoid arthritis, Alzheimer's disease, coeliac disease, multiple sclerosis, Guillane-Barre syndrome, neuritis, rheumatoid arthritis, synovitis, Sjogren's syndrome, Stiff Person Syndrom, myasthenia gravis, thryoiditis, systemic lupus erythematosus, lupus erythematosus, Addison's disease, pernicious anemia, Goodpasture's syndrome, Behcets's syndrome, allograft rejection, graft-versus-host disease, Type I diabetes, ankylosing spondylitis, Berger's disease, Type II diabetes, and Graves disease.

The extra boost administration(s) may be given in response to detecting symptoms of the disease or disorder.

It is also preferred that the autoantigen is formulated in an adjuvant, such as alum.

The present invention also includes a formulation, that may be used as pharmaceutical composition, comprising of a cocktail of sequestered autoantigens including at least one of GAD65, GAD67, Pro-insulin, Basic Myelin Protein, MOG, Chondrotoin II. The formulation may include an adjuvant, such as alum.

The subject of the present invention regards treatment of autoimmune disease. More specifically it includes specific upregulation of regulatory T cells.

GAD is a major autoantigen in autoimmune diabetes. GAD-alum was injected subcutaneously into 35 and placebo (alum) into 34 c-peptide positive, GADab+, recent onset type 1 diabetes patients aged 10-18 years at two occasions, day one and day 30. Alum was used as an adjuvant. After being followed for 15 months patients that received active drug showed a significantly higher ability to produce c-peptide after meal stimulation.

Not all patients responded, measured as improved c-peptide level compared to placebo however. The sooner the patients were treated once diagnosed the better were the results. This was a significant finding. Females possibly responded a little better than males. Older patients responded possibly a little better than younger.

Surprisingly however, when performing immunological mechanistic studies after 15 months it was found almost all if not all patients responded to stimulation with GAD. Almost no or none of the patients that received placebo responded immunologically. Fox P3 and the cytokine pattern that was obtained after stimulation with GAD after 15 months was in line with current thinking of a response that is needed for downregulation of autoimmune disease. Although triggered by GAD, the effect is not GAD specific. The measured increase in Fox P3 shows that the number of regulatory T cells are increased and it is known in the art that these regulatory T cells control the autoimmune response to autoantigens. As GAD, unlike many other endogenous proteins, is a sequestered autoantigen there is a pool of naïve T cells that can be recruited to respond to GAD. GAD in alum injected sc is shown to increase regulatory T cells. This proves that GAD-injections have a systemic effect and can be used to treat any autoimmune disease.

Based upon data recently obtained indicates that even those few patients who showed no decline in stimulated insulin production (measured as c-peptide) at 15 months started the decline of stimulated insulin production after 15 months. Accordingly, it is preferred that the treatment regimen will require more than just two doses. The recently obtained data suggests that added doses are preferably required to keep the insulin production up.

This data indicates that desirability of on-going, preferably regular treatment is preferred over vaccination alone.

Accordingly, the present invention includes a vaccination regimen and related formulations, and also includes a treatment regimen as well.

The treatment comprises a regimen wherein the patient receives a new treatment dose on a regular basis. Preferred interval between doses is 12 months, but more preferred every 9 months, preferably better every 6 months and most preferably at intervals of every 2nd to 5th month.

In accordance with the present invention it has been found that a two-dosage (i.e., two-shot) regimen, although effective, has an effect that is not as long-lasting as might be most desired. This may indicate that a treatment regimen is preferable over a vaccination regimen. Although not limited to the theory of the invention, the reason for this may be that when evoking active functional tolerance to an “endogenic” protein as one might do in a two shot regimen, the effect is not as lasting as it would have been if the protein was “exogenic.” Therefore the immune system needs to be constantly reminded in a regular treatment fashion (such as through regular boosts).

The present invention therefore also includes a method to treat autoimmune disease or disorder by administering at least one sequestered autoantigen in a prime and boost regimen for sensitization purposes followed by at least two boost dosages for treatment purposes.

As to timing of treatment dosages, it is preferred that the interval between the at least two boost dosages is less than 12 months (or more frequently as described above), preferably less than 9 months, less than 6 months, and most preferably in intervals of a duration of from about 2 to about 5 months. The boost dosages preferably are given at intervals of less than 12 months for a period of at least 2 years, preferably for longer periods of at least 5 or 10 years, and even over a substantial portion of a patient's lifespan.

The present invention also relates to medications and methods for treatment of autoimmune disease. In particular the present invention teaches medications, single-use prefilled syringes and vials containing the medication of suitable strength of sequestered endogenous autoantigens for three administrations within a six month period for patients diagnosed with autoimmune disease within six months prior to treatment. Moreover, the present invention teaches constructs, medications, formulations and methods to regulate inflammation in areas where sequestered autoantigens or epitopes thereof are found.

It is preferred that the formulations and methods of the present invention include at least one autoantigen selected from the group consisting of: GAD65, GAD67, Pro-Insulin, Basic Myelin Protein, MOG, Collagen Type II, ICA512 (IA2), ICA512B (IA2B), insulin, insulin B-chain, Hsp60, Hsp65, P277, ICA69, Glima38, SOX13, Imogen 38, Sulfatide, 21-Ohase, TPO, allergens, transplant antigens, cancer antigens, or parts, peptides or altered peptide ligands thereof.

The use of the medications and methods of the invention may be through administration via one or more routes including oral, nasal, inhaled, intramuscular, subcutaneous, intravenous, colorectal, transdermal, or by use of an implant or pump, use of DNA and RNA guns, or viral vectors such as AAV and HSV.

It is also preferred that the medications of the present invention contain constructs formulated in an adjuvant, such as aluminium hydroxide, when treating organ-specific autoimmune disease and an adjuvant that stimulates the cell-mediated arm of the immune system when treating cancer.

The formulations of the present invention are used for treating disease three times within a six month period from diagnosis and additional administrations are given in response to detecting symptoms of the disease or disorder, where preferably the formulation is used a fourth time within a 12 month period from diagnosis.

Generally, the present invention may be expressed broadly as a method for treating an autoimmune disease comprising the steps of (1) identifying a patient determined to have an autoimmune disease having progressed for not longer than 6 months, and (2) initiating treatment by administering an effective amount of a medicament for use to treat autoimmune disease within the 6 month period.

It is preferred that the treatment initiation occurs before the autoimmune disease has progressed for no longer than 3 months, and that the medicament is administered at least three times within 6 months from treatment initiation, and most preferably that both are the case.

In a preferred embodiment, in such instances the medicament is administered at least three times within 4 months from treatment initiation, more preferably at least three times within 3 months from treatment initiation; and most preferably at least four times within 10-12 months from treatment initiation. It is also preferred that the medicament is administered at least two times within 30 days from treatment initiation.

The method may be used against autoimmune disease characterized by antibody positivity to at least one antigen of the medicament and/or by abnormally high blood sugar levels. This may include type 1 diabetes.

The invention also includes a method for treating an autoimmune disease comprising the steps of (1) identifying a patient determined to have an autoimmune disease having progressed such that fasting C-peptide levels are greater than 0.2 pmol/ml, preferably having progressed such that fasting C-peptide levels are greater than 0.1 pmol/ml, and (2) initiating treatment by administering an effective amount of a medicament for use to treat autoimmune disease within the 6 month period from that point. It is preferred that patients be identified with fasting c.peptide levels above 0.1 pmol/ml and that treatment be initiated then before this fasting level goes below 0.1 pmol/ml, as the autoimmune disease attacks the insulin producing beta cells. When there is really almost no beta cells left the fasting C-peptide level goes below 0.1 pmol/ml. Typically, fasting c-peptide levels above a certain level (0.2 pmol/ml) leads to fewer long term complications.

The methods of the present invention may be used against autoimmune diseases characterized by using at least one major autoantigen being one from the constructs selected from the group consisting of GAD65, GAD67, Pro-Insulin, Basic Myelin Protein, MOG, Collagen Type II, ICA512 (IA2), ICA512B (IA2B), insulin, insulin B-chain, Hsp60, Hsp65, P277, ICA69, Glima38, SOX13, Imogen 38, Sulfatide, 21-Ohase, TPO, allergens, transplant antigens, cancer antigens, or parts, peptides or altered peptide ligands thereof.

It is preferred that the method be used such that the autoimmune disease is characterized by having the constructs formulated in a Th2-driving adjuvant, and that the Th2 driving adjuvant is alum.

The present invention may also use a medicament that enhances cell-mediated cytotoxic activity characterized by having the constructs selected from the group consisting of GAD65, GAD67, Pro-Insulin, Basic Myelin Protein, MOG, Collagen Type II, ICA512 (IA2), ICA512B (IA2B), insulin, insulin B-chain, Hsp60, Hsp65, P277, ICA69, Glima38, SOX13, Imogen 38, Sulfatide, 21-Ohase, TPO, allergens, transplant antigens, cancer antigens, or parts, peptides or altered peptide ligands thereof, and formulated in a Th1-driving adjuvant.

Examples of autoimmune diseases that may be treated by the method include cancer. Other autoimmune diseases may be selected from the group consisting of pancreatitis, pseudomembranous colitis, acute ulcerative colitis, chronic ulcerative colitis, achalasia, cholangitis, Crohn's disease, inflammatory bowl disease, enteritis, Whipple's disease, type 1 and type 2 diabetes, asthma, allergy, immune complex disease, organ ischemia, organ necrosis, hay fever, eosinophilic granuloma, granulomatosis, sarcoidosis, vaginitis, prostatitis, urethritis, bronchitis, emphysema, rhinitis, burns, dermatitis, dermatomyositis, urticaria, vasulitis, cardiovascular disease, atherosclerosis, pericarditis, myocarditis, myocardial ischemia, periarteritis nodosa, rheumatic fever, rheumatoid arthritis, Alzheimer's disease, coeliac disease, Multiple Sclerosis, Guillane-Barre syndrome, neuritis, rheumatoid arthritis, synovitis, Sjogren's syndrome, Stiff Person Syndrome, myasthenia gravis, thryoiditis, systemic lupus erythematosus, lupus erythematosus, Addison's disease, pernicious anemia, Goodpasture's syndrome, Behcets's syndrome, ankylosing spondylitis, Berger's disease, Graves disease, allograft rejection, graft-versus-host disease, and cancer.

The method may also use a medicament administered by at least one of the routes selected from the group consisting of oral, nasal, inhaled, intramuscular, subcutaneous, intravenous, colorectal, transdermal, or by use of an implant or pump, use of DNA and RNA guns, or viral vectors, such as amphions or defective AAV and HSV.

The method preferably uses an amount of at least one of the autoantigens that is between 10 and 150 micrograms per treatment occasion.

The present invention also includes medicament formulations as described, and as applied in the methods described herein.

### BRIEF DESCRIPTION OF THE DRAWINGS

Figure A is a flow diagram for patients in a study elucidating the present invention.

Figure B is a graph of the change in C-peptide from patients in a study elucidating the present invention.

Figure C is a graph of the change in C-peptide from patients in a study elucidating the present invention.

Figure D is a flow diagram for patients in a study elucidating the present invention.

Figure E shows changes in C-peptide levels in patients being treated within various periods of time after diagnosis.

FIGS. 1-19 are graphs of results of a study elucidating the present invention.

Figure A-21 is a flow diagram for patients in a study elucidating the present invention through the 21-month visit.

### DETAILED DESCRIPTION OF THE PREFERRED EMBODIMENT

In accordance with the foregoing summary, the following present a detailed description of a preferred embodiment of the present invention which is currently considered to be the best mode thereof.

The present invention may be further appreciated from the following study of patients.

### Methods

The study was approved by the research ethics committee at Linkoping University, Sweden, and by the regulatory authorities in Sweden.

### Study Design

At eight pediatric clinics in Sweden, 10-18 year old T1D patients who had presented with disease within the previous 18 months were screened for presence of GAD65 autoantibodies (GADA) and fasting C-peptide levels above 0.1 pmol/ml. A total of 70 patients were eligible and randomized to a double blind treatment of either 20 μg of recombinant human GAD65 formulated in alum (Diamyd®, Diamyd Medical, Stockholm, Sweden; 35 patients) or placebo (the same formulation without rhGAD65; 35 patients).

All patients were treated with Multiple Insulin Therapy and both the patients and their parents or guardians provided informed consent. The trial objective was to evaluate the safety as well as the efficacy of treatment compared to placebo in preserving residual insulin secretion. The primary efficacy endpoint was change in fasting C-peptide level from baseline to month 15. The secondary efficacy endpoints were changes from baseline in stimulated C-peptide levels and HbA1c.

Each patient received a subcutaneous primary injection of either GAD65 or placebo on day 1 followed by a boost one month later. Patients remained in the clinic to be observed for three hours after injection.

On day 1 and at months 3, 9 and 15, a mixed meal tolerance test (MMTT) was performed in accordance with the European study on estimation of beta cell function²⁵ which includes ingestion of 6 ml Sustacal®/kg body weight (Sustacal®, Mead Johnson, Evansville, Ind., USA). Blood samples for C-peptide analysis were collected before, 30, 60, 90 and 120 minutes after beginning of the MMTT. Safety evaluations, including neurological assessments, clinical examination, hematology, biochemistry, and impact of treatment on diabetes status were repeatedly assessed throughout the study.

After completion of the main study period (15 months), the treatment code was opened, and data analyzed including C-peptide levels (fasting, max, area under the curve [AUC]), HbA1c, blood glucose, insulin requirement (units per kg body weight and 24 hours) and GADA titre.

With only the statistician, the SAS programmer and the sponsor being informed of unblinded data, the study continues in a partly blinded fashion for an extension period of 15 months and additional MMTTs will be performed at 21 and 30 months.

### Laboratory Tests

Laboratory analyses were performed at Linkoping University, Sweden. C-peptide levels were measured in serum samples with a Time-resolved fluoroimmunoassay (AutoDELFIA™ C-peptide kit, Wallac, Turku, Finland). Results were validated with inclusion of a C-peptide control module containing a high, a medium and a low-level control in each assay (commercially available from Immulite, DPC, UK). A 1224 MultiCalc® program (commercially available from Wallac) was used for automatic measurement and result calculation and measurements were expressed in pmol/ml.

Serum GADA titres were determined in duplicate using a radio binding assay employing 35S-labelled recombinant human GAD65 produced by in vitro transcription/translation (pEx9 vector kindly supplied by Prof. Ake Lernmark, University of Washington, Seattle, Wash., USA). Sepharose protein A was used to separate free from antibody bound labeled GAD65. GADA levels are presented with a maximum of 500 U/ml as it was decided to determine maximal titres at the end of the study. HLA-DQ A1* and B1* alleles were determined by PCR amplification of exon 2 sequences and hybridization with allele-specific probes detected by time resolved fluorescence as described.²⁶ As detailed in a population-based Swedish case-control study²⁷, the patients were then divided into very high risk, high risk, moderate risk, neutral and low risk subjects.

### Statistical Analysis

Results from a previous study in LADA-patients²⁴ suggested that 35 patients in each treatment group would provide a power of 80-90% for assessing differences in C-peptide levels, with a significance level of 5%; assuming a mean difference of 0.12 pmol/ml and a standard deviation of 0.15 in fasting C-peptide levels. Data management and the statistical analysis were performed by Trial Form Support AB, Lund, Sweden. An analysis of covariance (ANCOVA) model was used where the change from baseline to month 15 was used as response variable, treatment as explanatory variable and baseline value as a covariate. Factors such as age, gender, duration of diabetes at intervention, GADA titre, and HLA type were identified in advance as possible factors for additional exploratory analyses.

In all tests, the null hypothesis was that there is no difference between active treatment and placebo. For all tests two-sided hypotheses were used and the p-values presented together with 95% confidence intervals. As there is only one primary analysis, the p-values are not adjusted for multiplicity.

### Results

### Recruitment and Randomization

Of 118 patients screened, 42 girls and 28 boys were eligible. The screening took place over two weeks in January and February 2005. The first patient injection was in February 2005, and the last patient completed the 15-month visit in July 2006.

All but one patient received two doses of either GAD65 or placebo (Figure A). One patient (girl, placebo) was withdrawn from the study due to mononucleosis with icterus and received only one injection. Sixty-nine patients, 35 Diamyd® treated and 34 placebo, were included in the per protocol analysis. No “intention-to-treat” analysis was performed as only one patient (placebo) dropped out.

TABLE 1
Baseline demographic and clinical characteristics of treatment groups

| | Diamyd ® | Placebo |
|---|---|---|
| Characteristic | (n = 35) | (n = 34) |
| Mean age ± SD, years | 13.8 ± 2.3 | 12.8 ± 1.9 |
| Mean duration of diabetes ± SD, | 9.9 ± 5.3 | 8.8 ± 5.4 |
| months | | |
| Mean BMI ± SD, kg/m2 | 19.5 ± 2.4 | 20.5 ± 3.2 |
| Gender distribution, n (%) | | |
| Female | 23 (66) | 18 (53) |
| Male | 12 (34) | 16 (47) |
| HLA classification, n (%) | | |
| Very High Risk (VH) | 8 (23) | 9 (26) |
| High Risk (H) | 10 (29) | 7 (21) |
| Moderate Risk (M) | 9 (26) | 7 (21) |
| Neutral (N) | 4 (11) | 4 (12) |
| Puberty stage at screening (Tanner Genital | 4 (11) | 7 (20) |
| Organs Stage), n (%) | | |
| Stage 1 | 8 (23) | 10 (29) |
| Mean insulin dose/kg | 0.66 ± 0.30 | 0.66 ± 0.28 |
| bodyweight ± SD, U/kg | | |
| Mean blood glucose prior to | 9.4 ± 4.0 | 8.8 ± 3.3 |
| MMTT ± SD, mmol/l | | |
| Mean HbA1c ± SD, % | 6.3 ± 1.3 | 6.2 ± 1.0 |
| Mean fasting C-peptide ± SD, | 0.33 ± 0.19 | 0.35 ± 0.23 |
| pmol/ml | | |
| Mean stimulated C-peptide Maximum ± | 0.78 ± 0.36 | 0.86 ± 0.54 |
| SD, pmol/ml | | |
| Mean stimulated C-peptide AUC ± SD, | 1.24 ± 0.57 | 1.41 ± 0.87 |
| pmol/ml * 2 hours | | |
| Median GADA titre, U/ml | ≧500 | ≧500 |
| GADA titre <500 U/ml, n (%) | 15 (43) | 11 (32) |
| GADA titre ≧500 U/ml, n (%) | 20 (57) | 23 (68) |

### Baseline Characteristics

Baseline data (baseline=day of first injection, prior to injection) shows that the two treatment groups were similar in most aspects (Table 1). The distribution of HLA genotypes did not differ between the Diamyd® and the placebo group (Table 1).

### Safety

There were no treatment related serious adverse events in the study. An equal number of mild skin reactions (erythema, edema and tenderness) was observed at the injection site in the Diamyd® and the placebo groups. None required treatment or led to refusal of the second injection. Neurological assessment, based on the potential concern of inducing stiff person syndrome, indicated no difference between the study groups.

### Efficacy

Both treatment groups showed a progressive decrease from baseline regarding both fasting and stimulated C-peptide secretion, indicating a gradual loss of beta cell function. There was no significant effect on fasting C-peptide (Table 2). However, over the 15 months, stimulated C-peptide secretion, as measured by AUC, decreased only half as much in the Diamyd® treated group as in the placebo group (p=0.01) (Figure B and Table 2). Also, maximum stimulated C-peptide deteriorated significantly less in the Diamyd® treated group (p=0.04) (Table 2).

### Effect on Diabetes Status

Both treatment groups increased their insulin requirement, blood glucose and HbA1c level during the study. As for C-peptide, all these parameters changed less in Diamyd® compared to placebo patients (Table 2).

Diamyd® treated patients tended to have a GADA titre above 500 after 15 months more often compared to placebo (Table 3). HLA genotype neither affected the baseline nor the change in C-peptide AUC during the 15 month period (Table 5).

TABLE 2
Endpoints, absolute values and mean change from baseline to month 15
(Absolute C-peptide values may be added into this Table)

| | | | Treatment | |
|---|---|---|---|---|
| Mean change in characteristic ± | Diamyd ® | Placebo | Effect* | p-value |
| SD | (n = 35) | (n = 34) | (95% C.I.) | (ANCOVA) |
| ΔFasting C-peptide, pmol/ml | −0.12 ± 0.18 | −0.17 ± 0.20 | 0.04 (−0.04, 0.12) | 0.28 |
| ΔStimulated C-peptide Maximum, | −0.24 ± 0.26 | −0.42 ± 0.40 | 0.16 (0.01, 0.31) | 0.04 |
| pmol/ml | | | | |
| ΔStimulated C-peptide AUC, | −0.38 ± 0.46 | −0.75 ± 0.61 | 0.30 (0.07, 0.54) | 0.01 |
| pmol/ml * 2 hour | | | | |
| ΔInsulin dose/kg bodyweight, U/kg | 0.15 ± 0.22 | 0.22 ± 0.29 | −0.08 (−0.19, 0.04) | 0.19 |
| ΔBlood glucose prior to MMTT, | 0.1 ± 5.8 | 1.3 ± 5.8 | −0.6 (−2.8, 1.6) | 0.57 |
| mmol/l | | | | |
| ΔHbA1c, % | 0.3 ± 1.3 | 0.5 ± 1.5 | −0.2 (−0.7, 0.4) | 0.57 |

*Treatment effect estimated using least square means methodology with baseline value as covariate.

TABLE 3
GADA titre at month 15

| | Diamyd ® | Placebo | p-value |
|---|---|---|---|
| | (n = 35) | (n = 34) | (Fisher's Exact |
| Median GADA titre, U/ml | ≧500 | ≧500 | — |
| GADA titre <500 U/ml, n (%) | 7 (20) | 14 (41) | 0.07 |
| GADA titre ≧500 U/ml, n (%) | 28 (80) | 20 (59) | |

### Exploratory Analyses and Interaction

Out of the patients who had a maximum stimulated C-peptide above 0.2 pmol/ml at baseline, only 19% of the Diamyd® treated patients fell below that limit by month 15 compared to 42% of the placebo patients (Table 4).

TABLE 4
Patients with a maximum stimulated C-peptide >0.2 pmol/ml

| | Diamyd ® | Placebo |
|---|---|---|
| Visit | (n = 35) | (n = 34) |
| Baseline, n (% of baseline value) | 32 (100) | 33 (100) |
| Month 15, n (% of baseline value) | 26 (81) | 19 (58) |
| Change, n (% of baseline value) | −6 (−19) | −14 (−42) |

Covariate analyses of the changes in fasting and stimulated C-peptide from baseline to month 15 across the 69 patients were performed using baseline C-peptide and treatment as covariates together with either duration of diabetes, age, gender, or baseline GADA levels. These analyses showed that only baseline C-peptide levels and treatment with Diamyd® had a statistically significant effect on residual insulin secretion during follow up (data not shown).

The subgroups specified in the protocol regarding duration of diabetes, age, gender, baseline GADA levels and HLA classification were investigated for effects on the efficacy of GAD65. For this purpose the efficacy was defined as the lessening of the deterioration in AUC from baseline until month 15 in the Diamyd® group compared to placebo. No formal statistical analysis was conducted due to small sample sizes and potential issues of multiple comparisons (Table 5).

In all tests, the null hypothesis was that there is no difference between active treatment and placebo The preservation of residual insulin secretion was more pronounced among patients treated shortly after T1D onset (Figure C, Table 5).

TABLE 5
Mean change ± SD in stimulated C-peptide AUC from
baseline to Month 15 for sub groups

| Sub group | Diamyd ® | | | Placebo | | |
|---|---|---|---|---|---|---|
| (classification | (pmol/ml * 2 hour) | | | (pmol/ml * 2 hour) | | |
| at baseline) | n | Baseline | Change | n | Baseline | Change |
| Duration | | | | | | |
| 0-3 months | 4 | 1.52 ± 0.34 | +0.10± | 7 | 1.63 ± 0.55 | −0.89± |
| 3-6 months | 7 | 1.49 ± 0.54 | −0.53± | 7 | 1.72 ± 1.24 | −1.08± |
| 6-12 months | 7 | 0.90 ± 0.49 | −0.46± | 9 | 1.48 ± 0.88 | −0.71± |
| 12-18 months | 17 | 1.21 ± 0.61 | −0.40± | 11 | 1.03 ± 0.70 | −0.44± |
| Age | | | | | | |
| 10-12 years | 11 | 1.21 ± 0.56 | −0.50± | 18 | 1.30 ± 0.71 | −0.69± |
| 13-15 years | 15 | 1.17 ± 0.58 | −0.36± | 13 | 1.28 ± 0.72 | −0.72± |
| 16-18 years | 9 | 1.38 ± 0.61 | −0.27± | 3 | 2.68 ± 1.52 | −1.15± |
| Gender | | | | | | |
| Females | 23 | 1.32 ± 0.66 | −0.34± | 18 | 1.56 ± 0.95 | −0.91± |
| Males | 12 | 1.08 ± 0.31 | −0.45± | 16 | 1.25 ± 0.76 | −0.57± |
| GADA titre | | | | | | |
| <500 U/ml | 15 | 1.25 ± 0.50 | −0.52± | 11 | 1.18 ± 1.11 | −0.77± |
| ≧500 U/ml | 20 | 1.23 ± 0.63 | −0.27± | 23 | 1.53 ± 0.72 | −0.73± |
| HLA | | | | | | |
| classification | | | | | | |
| Very High Risk | 8 | 1.02 ± 0.74 | −0.33± | 9 | 1.48 ± 0.77 | −0.85± |
| High Risk | 10 | 1.09 ± 0.51 | −0.26± | 7 | 1.39 ± 0.88 | −0.98± |
| Moderate Risk | 9 | 1.56 ± 0.54 | −0.63± | 7 | 1.54 ± 1.22 | −0.88± |
| Neutral Risk | 4 | 1.38 ± 0.34 | −0.48± | 4 | 0.89 ± 0.63 | −0.16± |
| Low Risk | 4 | 1.19 ± 0.43 | −0.10± | 7 | 1.53 ± 0.81 | −0.53± |

### Discussion

No significant beneficial effect was found in fasting C-peptide, which was chosen as a primary endpoint based on the previous study in LADA patients.²⁴ However, fasting C-peptide levels in recent onset T1D patients may remain near normal when fasting glucose is kept low because of active treatment³⁰ while stimulated C-peptide levels decline over time as a consequence of continuous beta cell destruction. This means that early in the course of the disease, stimulated C-peptide is a preferred endpoint for assessing preservation of beta cell function, which is correlated with improved glycemic control and less micro-vascular complications.³¹

The results demonstrate that two injections of 20 μg GAD65 significantly improves preservation of residual insulin secretion in patients with recent onset T1D during a follow-up time of similar length as in other intervention studies (14,16,17). In the responding patients, mainly newly-diagnosed, the effect size and duration is similar with GAD65 as with antiCD3 (16,17), but without adverse events after GAD65 treatment Residual insulin secretion affects important clinical outcome parameters. Furthermore, a smaller proportion of patients in the GAD65 than in the placebo group lost their capacity to secrete more than 0.2 pmol/ml of C-peptide in response to a meal. In the Diabetes Control and Complications Trial (DCCT), patients with less than 0.2 pmol/ml in stimulated C-peptide had a higher risk for retinopathy and severe hypoglycemia than patients above that threshold.^{5,28} This may be explained by improved overall metabolic control, less blood glucose fluctuations and possibly, by greater C-peptide exposure, which itself may have biologic effects.²⁹ To our knowledge there are few clinical trials with an immunomodulation approach that have achieved a reduced loss of residual insulin secretion as in the present study.

Both treatment groups increased their insulin requirement, blood glucose and HbA1c level during the study. However, all three parameters showed a reduced, but statistically non-significant, increase in the Diamyd® compared to the placebo group. A tendency to increase in GADA levels was seen in the drug treated group which was expected as GAD65 was injected in an immunomodulatory vaccine formulation. The effect on other auto-antibodies as well as on cell-mediated immunity will be subject to further analysis.

Small and insignificant differences in the demographics of the drug treated patients and the placebo patients are unlikely to be relevant. While the drug treated patients were slightly older, the placebo patients had a somewhat shorter disease duration and higher residual insulin secretion on average at intervention. The distribution of HLA genotypes were comparable and did not explain differences between the Diamyd® and placebo groups. It was important to carefully evaluate the possible role of HLA as it has been shown that GADA in T1D patients were associated with HLA-DQA1*0501-B1*02 in T1D patients.²⁷ Further studies in larger number of patients will be needed to evaluate the possible importance of HLA genotypes in response to Diamyd®.

As a previous study had suggested effect of GAD85 vaccination in LADA patients with slowly progressive autoimmune diabetes, we chose to include not only newly-diagnosed T1D patients, but those with a duration up to 18 months. However, the protective effect of Diamyd® on stimulated C-peptide tended to be especially pronounced in patients treated shortly after diagnosis. Our design makes subgroup analyses difficult, but, if confirmed, patients with short disease duration and good residual insulin secretion might improve their function enough to go into complete remission, were the ongoing autoimmune attack abated by the treatment.

The mechanism of how Diamyd® treatment altered disease progression in T1D patients is not yet clear. The same type of insulin treatment in all patients, and the small difference in HbA1c should exclude that the preserved beta cell function is the consequence of more intense insulin treatment or better metabolic control in the GAD65 group. Numerous animal studies in NOD mice have shown that GAD65 can induce potent regulatory response in mice with established autoimmunity and after the onset of T1D.^{22,23} It is of interest in this regard that the prior dose-finding study revealed that Diamyd® induced an increase in the (CD4+CD25+)/(CD4+CD25−) cell ratio at 24 weeks suggesting a possible effect on regulatory (CD4+CD25+) T cells.²⁴

In conclusion, the treatment with alum-formulated GAD65 had a protective effect on residual beta cell function 15 months after intervention. The demonstrated efficacy of GAD65, in line with or better than other developing treatments reported so far, gives hope that autoantigen-specific immunomodulation, alone or used in combination therapy, may eventually prevent T1D. It is promising that the protective effect on beta cell function in this study occurred with the use of a drug which is very easy to administer and well tolerated with mild and few adverse events, not differing from placebo.

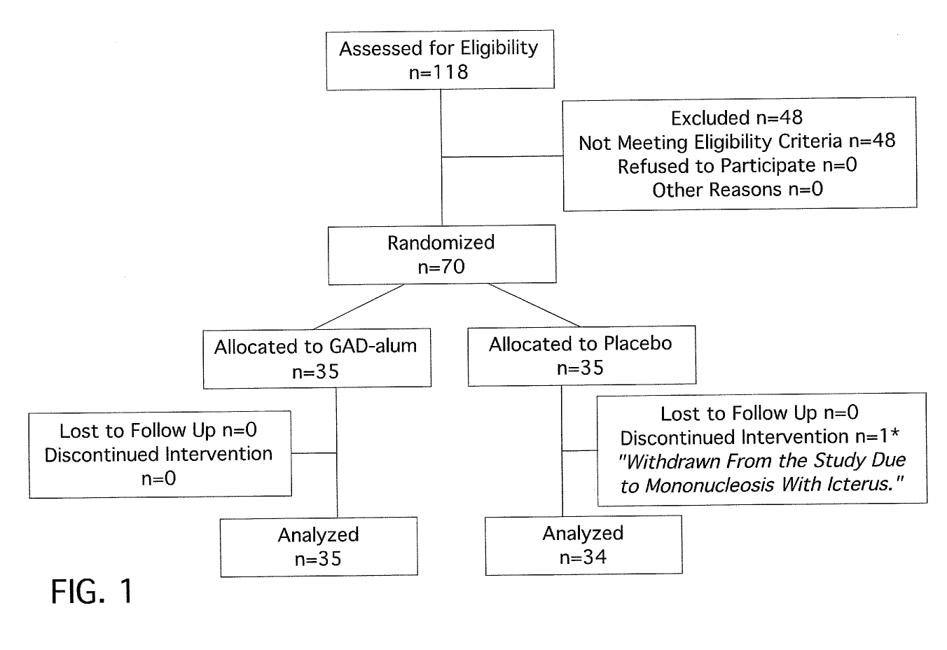

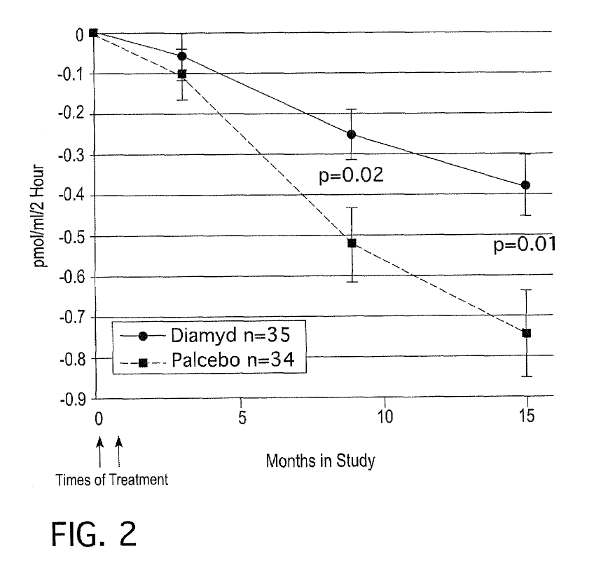

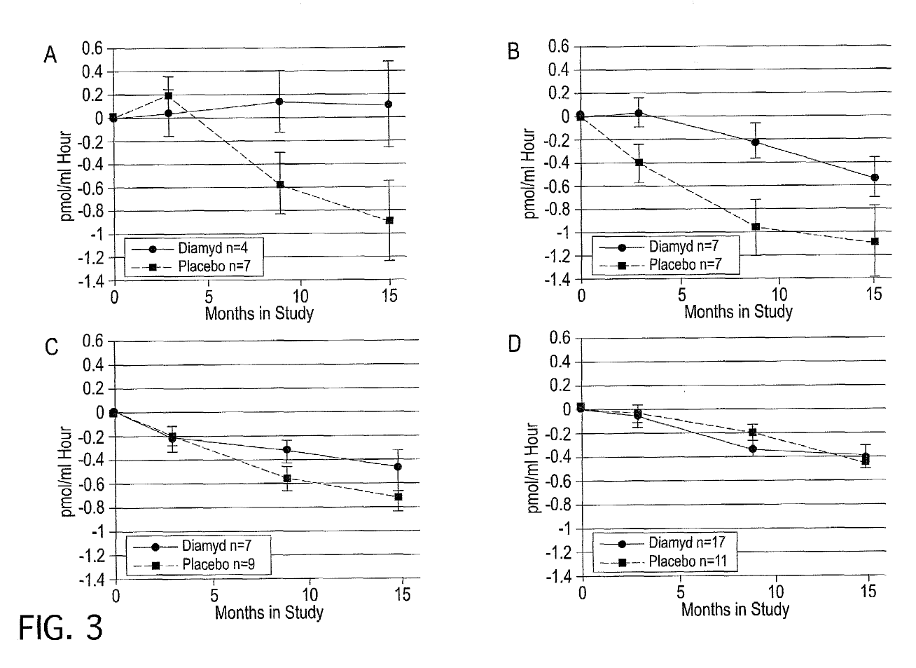

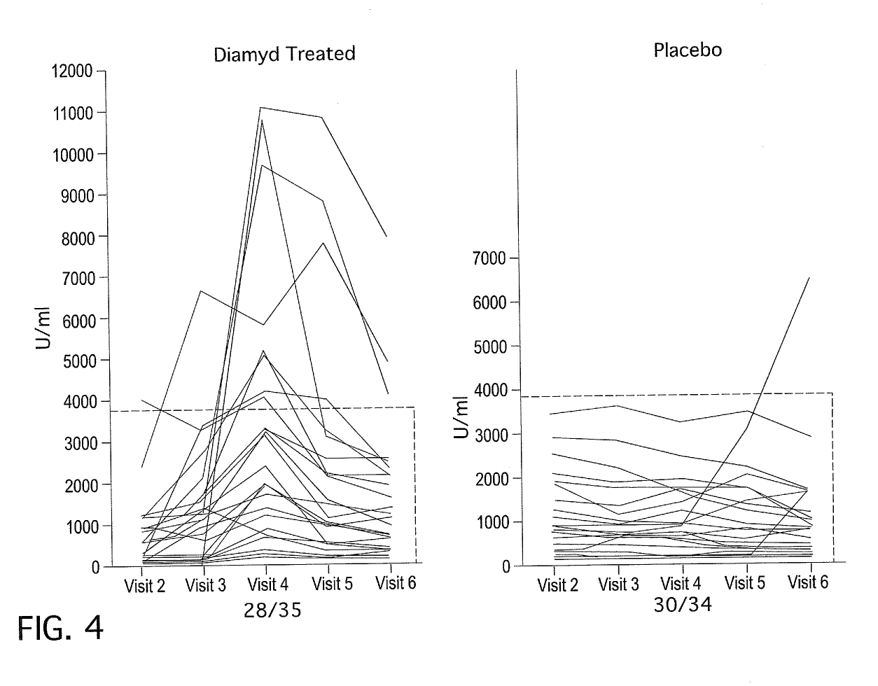

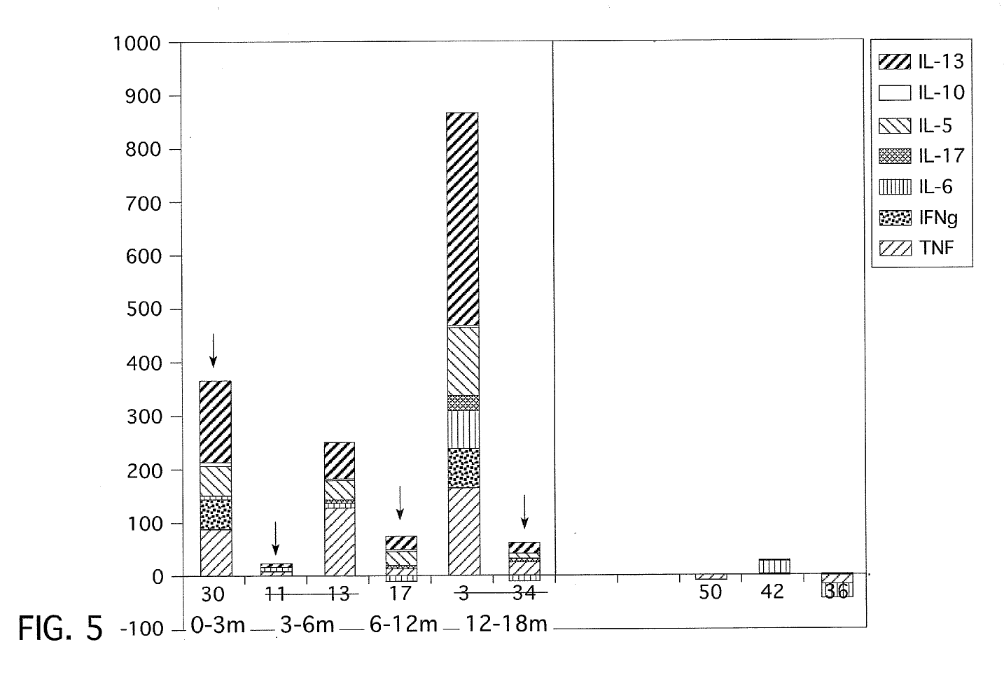

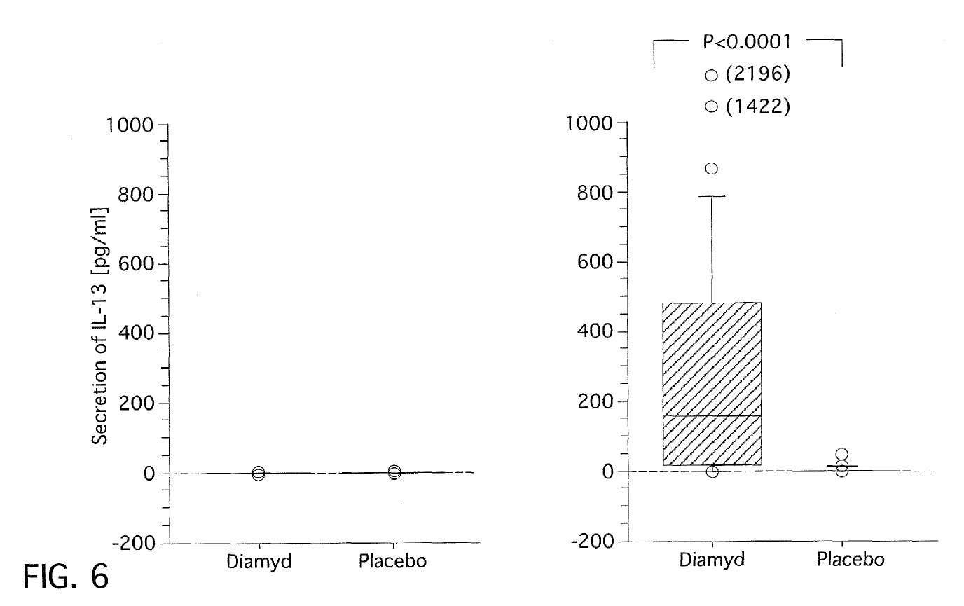

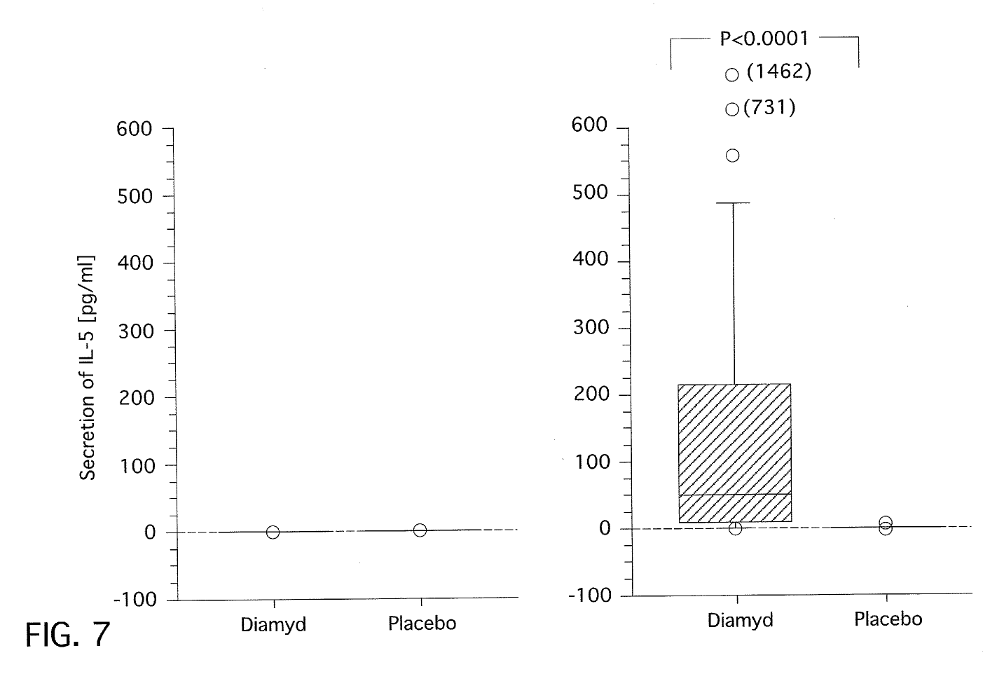

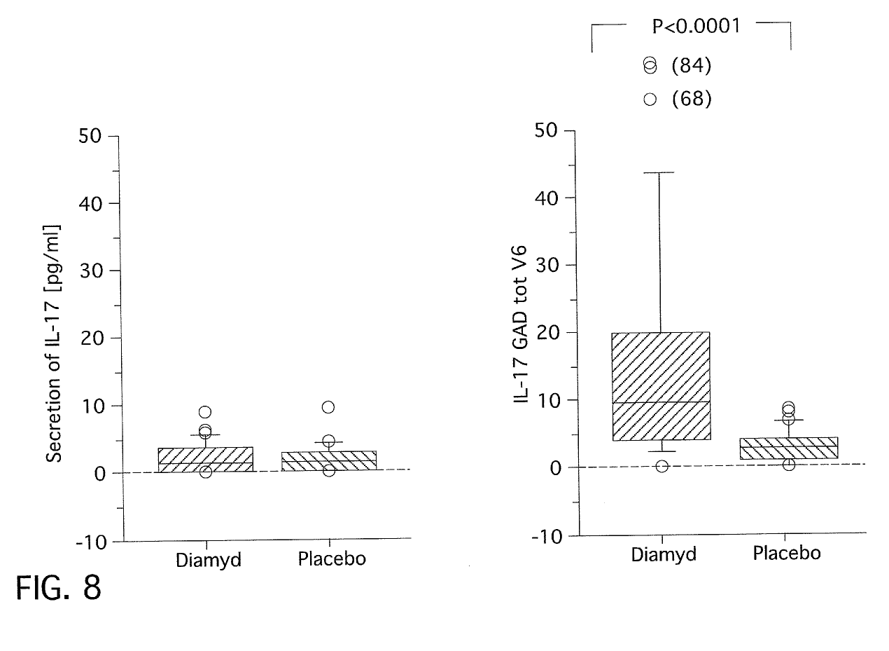

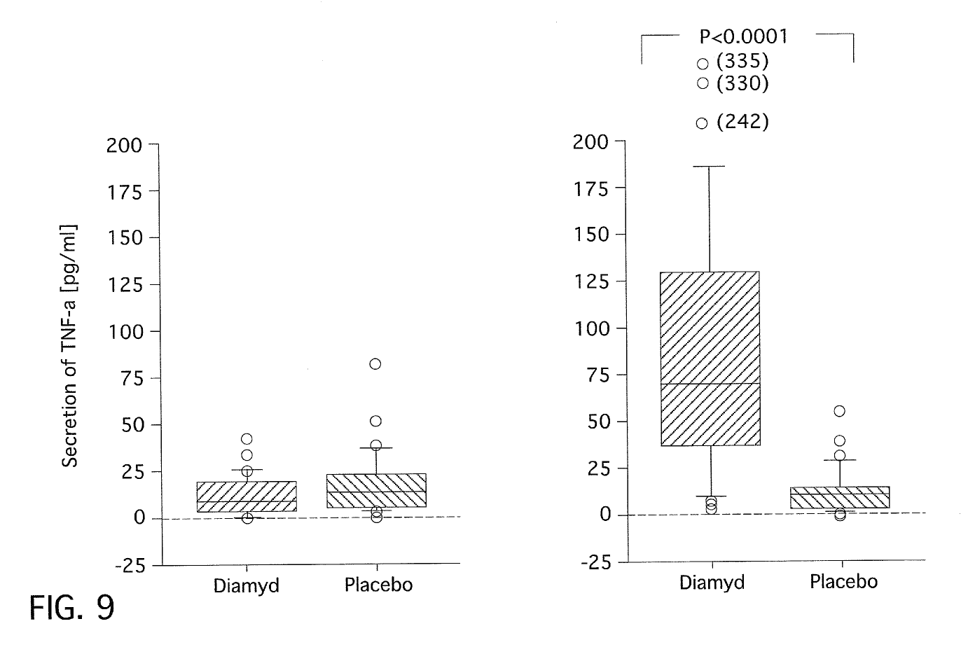

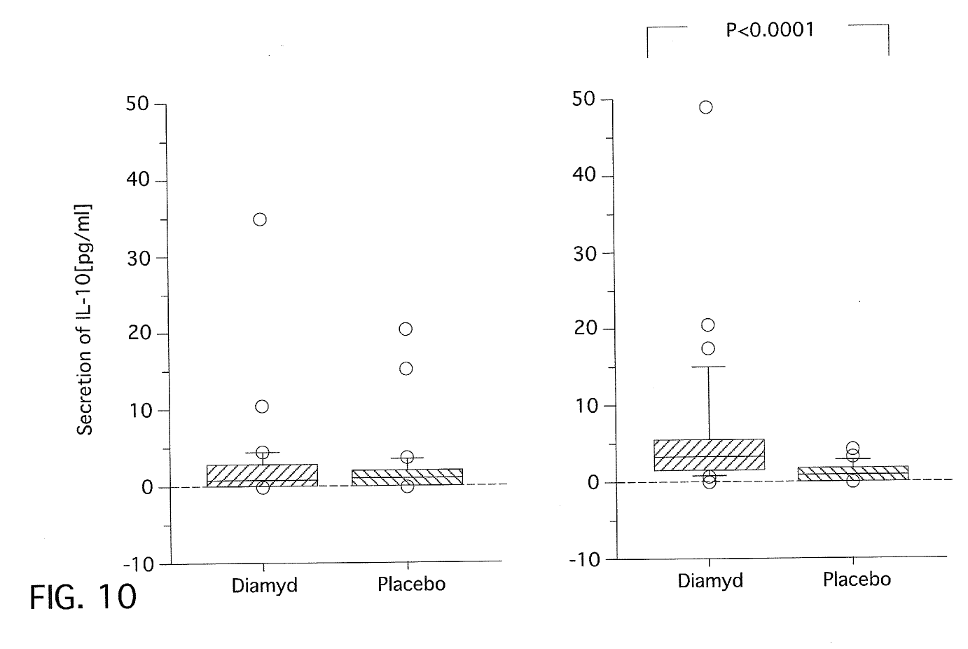

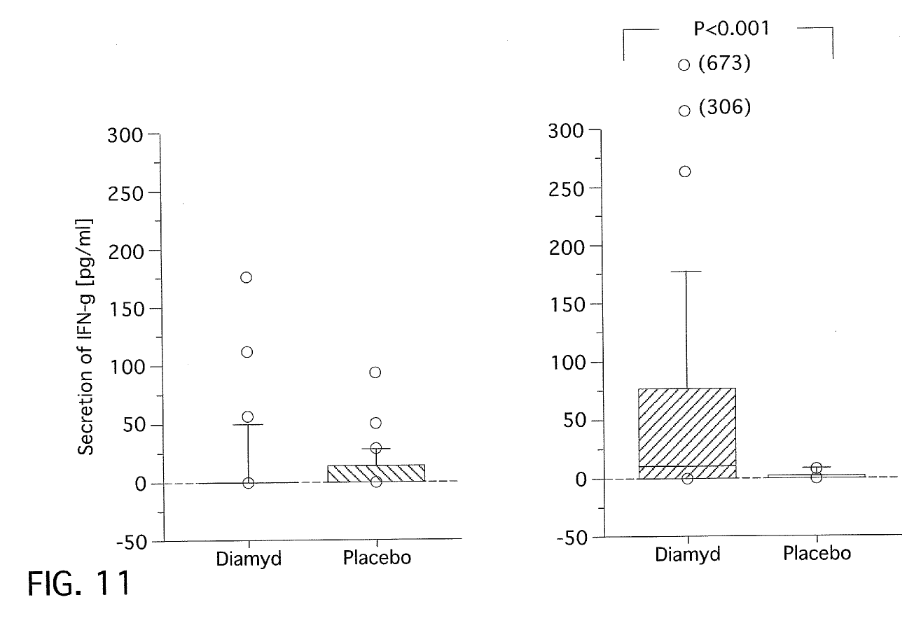

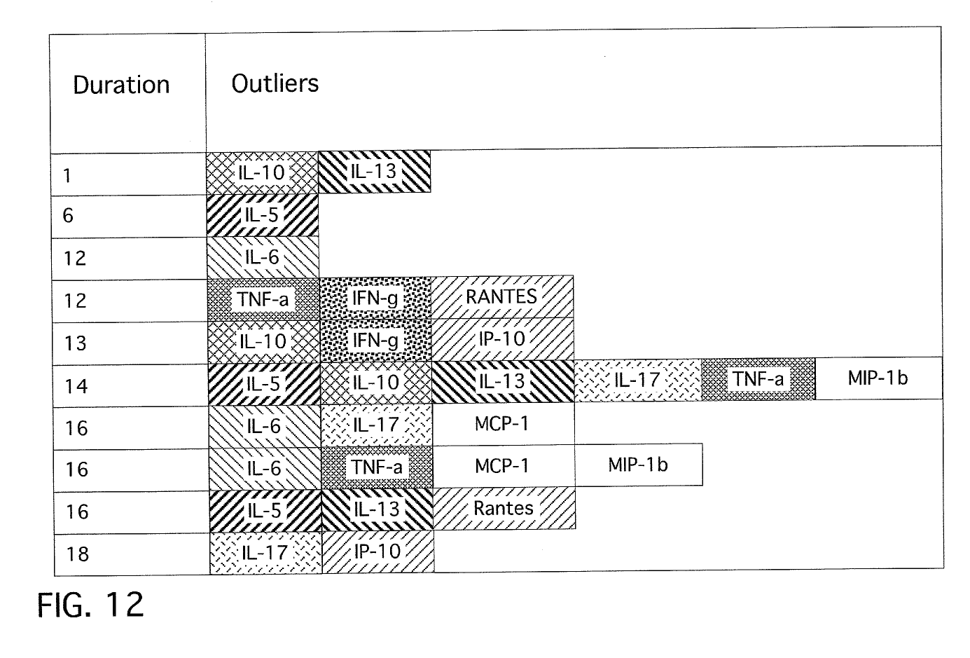

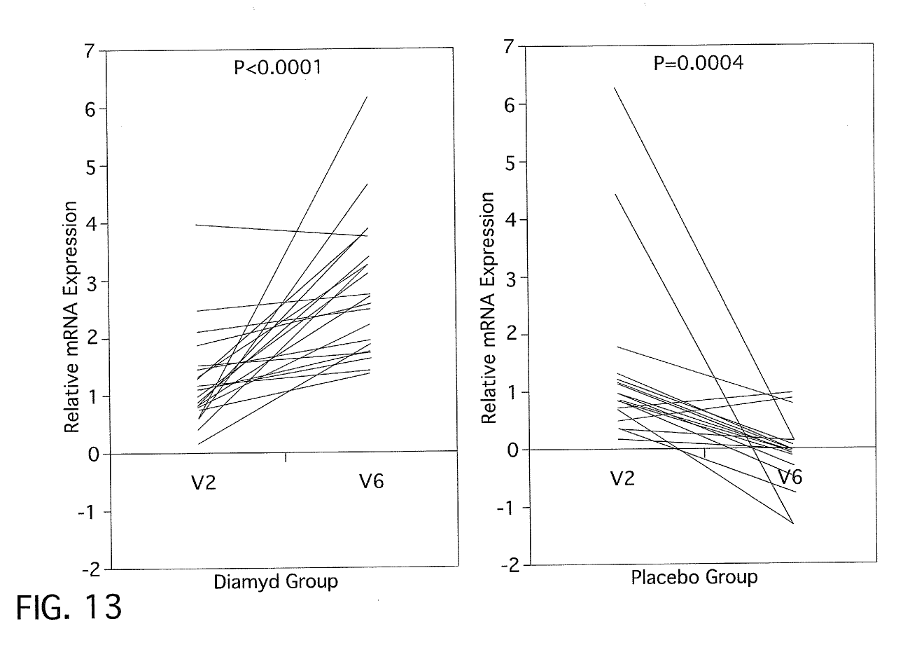

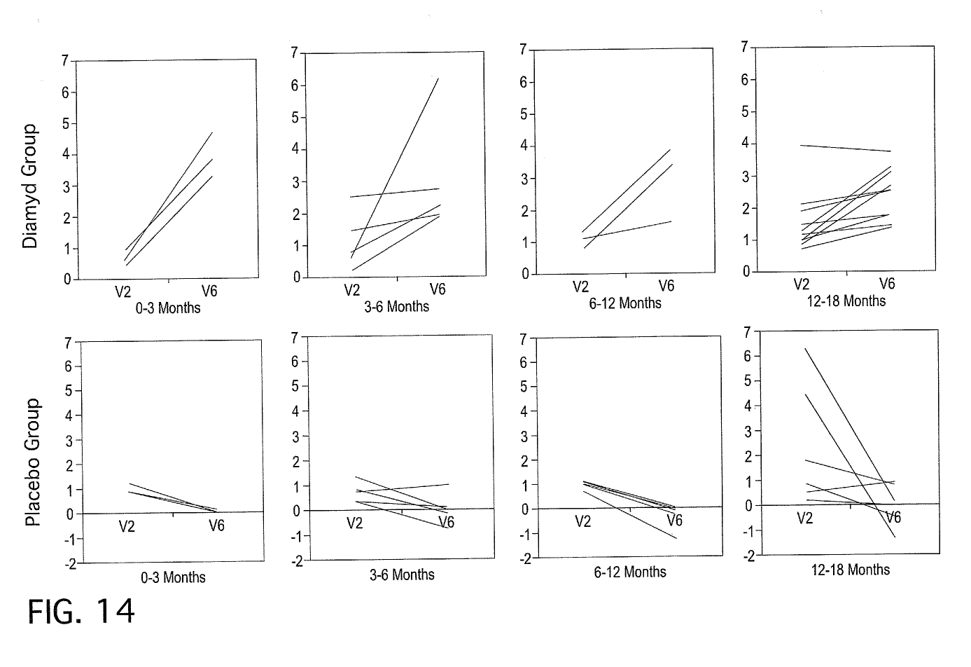

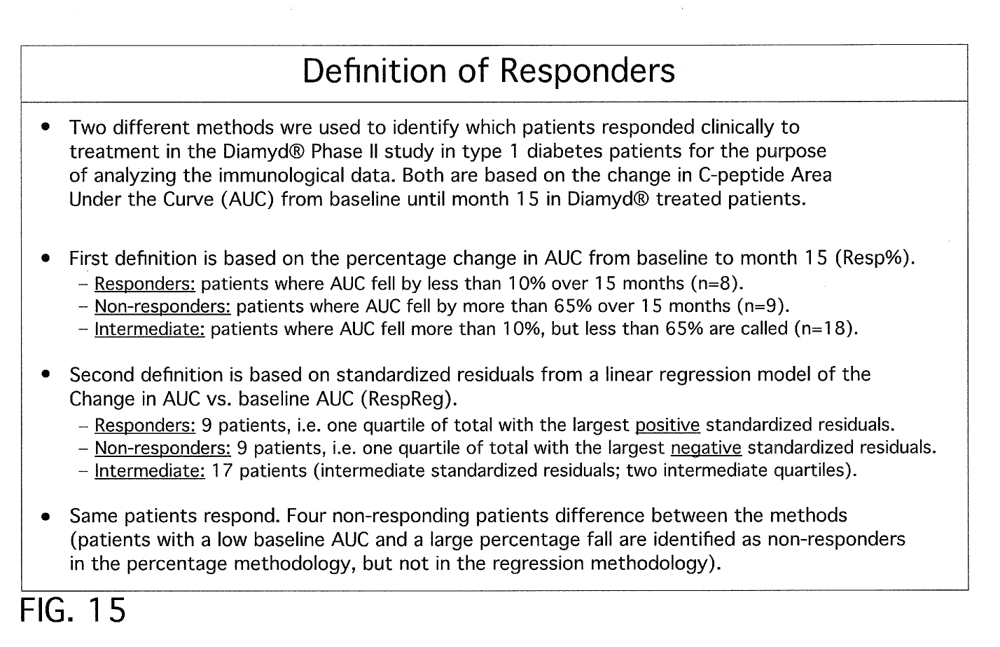

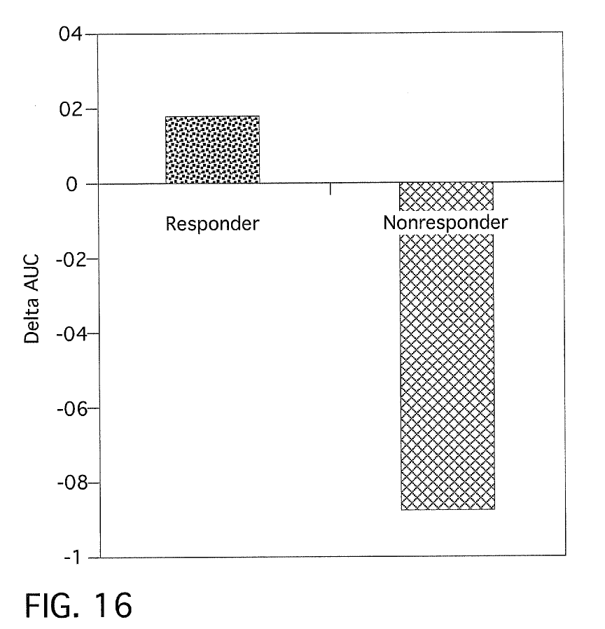

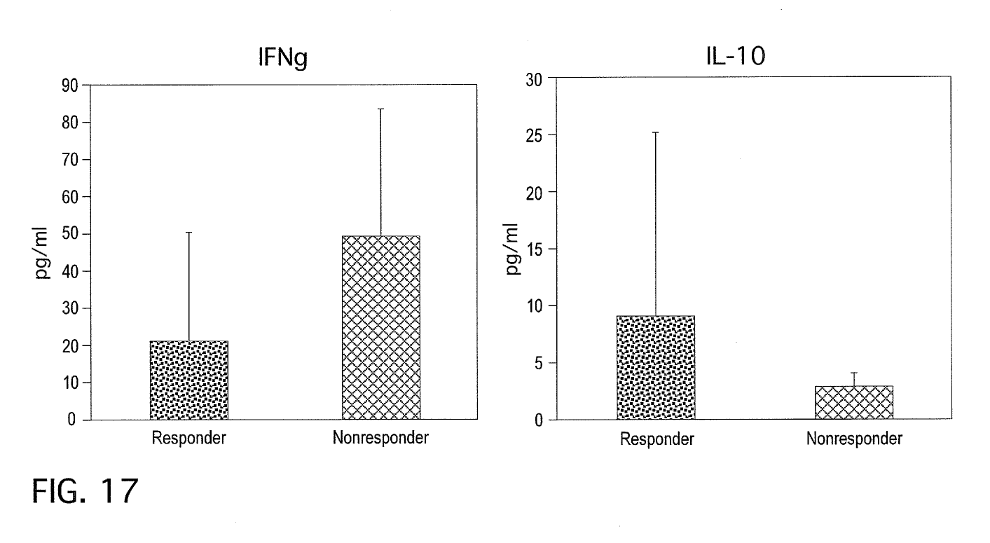

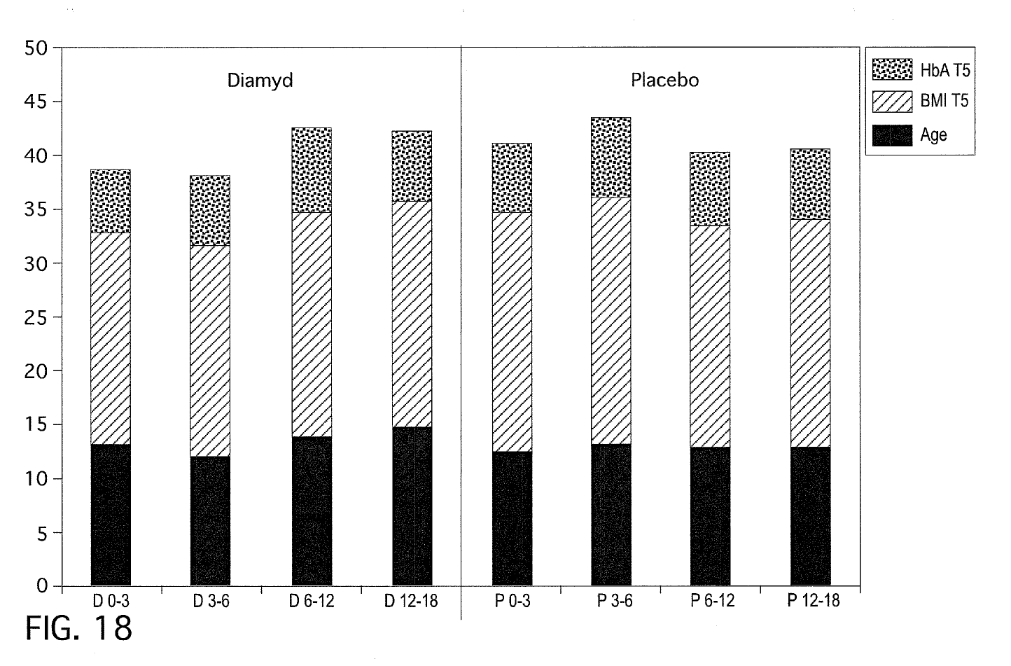

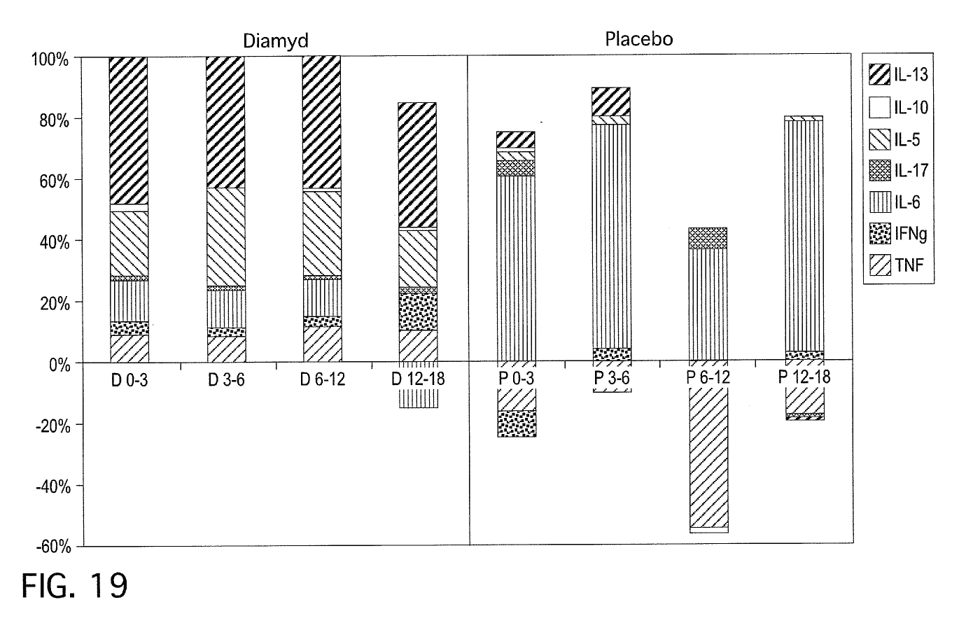

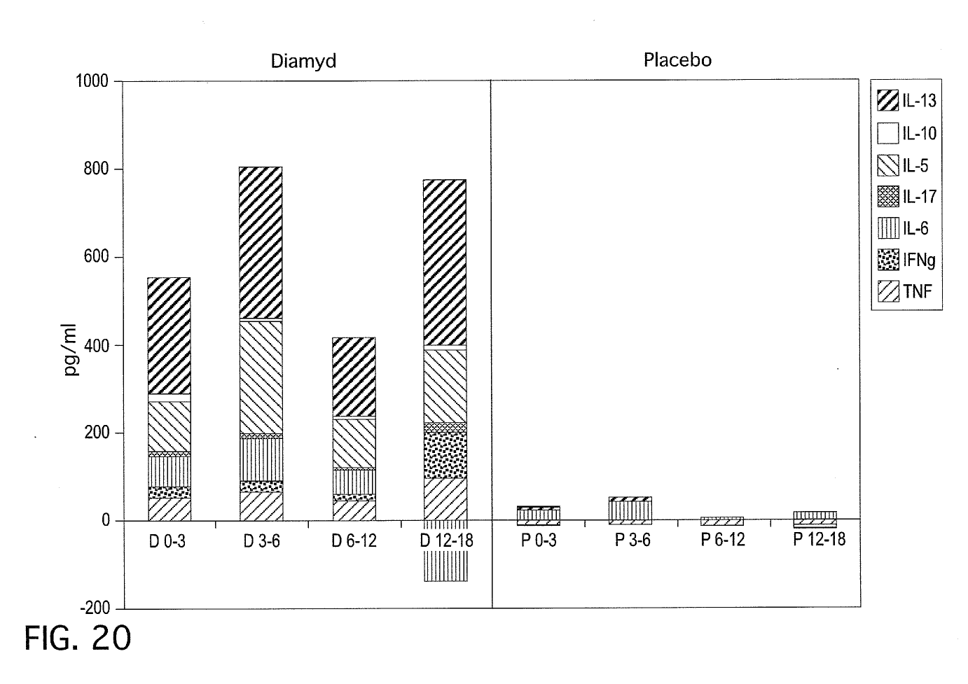

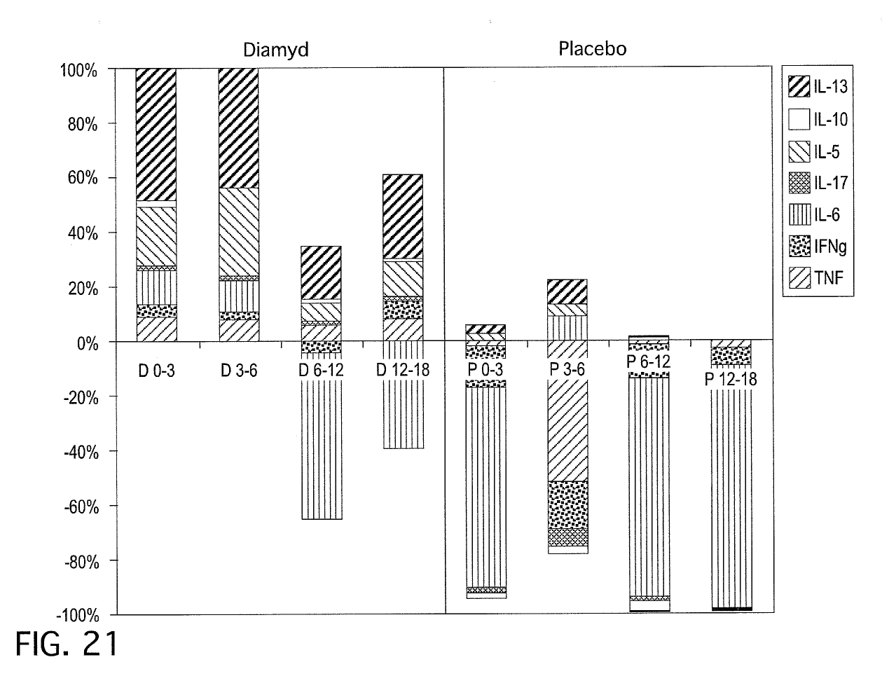

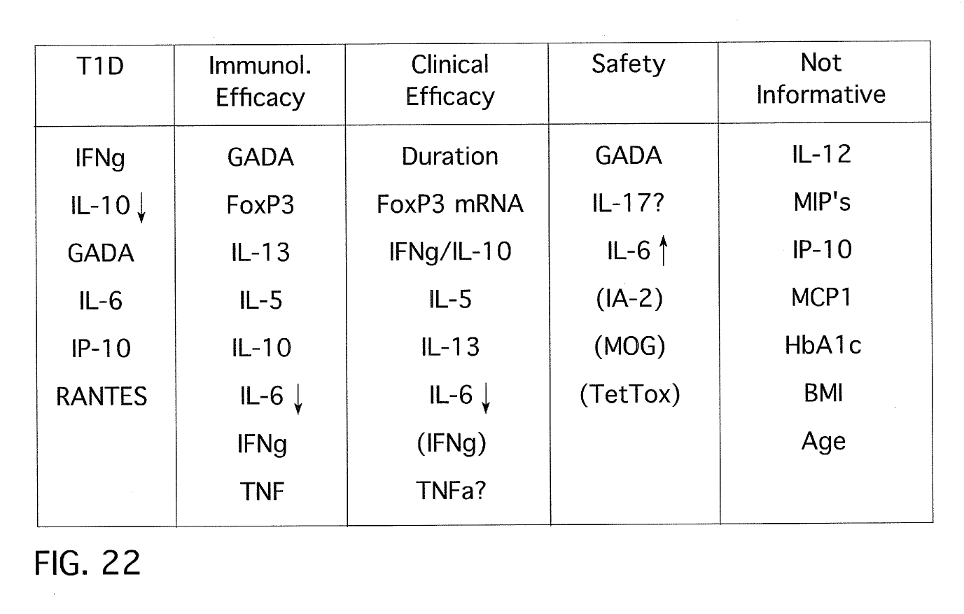

FIGS. 1-17 are graphs of results of a study elucidating the present invention.

In addition, the foregoing study, using the same methods and study design described herein, has been continued to determine whether administration of GAD-alum in young T1D patients of recent onset was safe and could reduce or halt the loss of residual insulin secretion. The following reports the results after a 21-month study period.

With only the statistician, the SAS programmer and the sponsor being informed of unblinded data, the study continues in a partly blinded fashion for an extension period of 15 months with MMTTs performed at 21 and 30 months. Here we report data from the 21-month follow-up.

HbA1c was analyzed by an immunological method, calibrated against the Swedish national standard Mono-S and continuously controlled against the External Quality Assurance in Laboratory medicine in Sweden (EQALIS) reference method.

### Further Statistical Analysis

Continuing the study as described above, results from a previous study in LADA-patients²⁴ suggested that 35 patients in each treatment group would provide a power of 80-90% for assessing differences in C-peptide levels, with a significance level of 5%; assuming a mean difference of 0·12 pmol/ml and a standard deviation of 0·15 in fasting C-peptide levels. Data management and the statistical analysis were performed by Trial Form Support AB, Lund, Sweden. An analysis of covariance (ANCOVA) model was used where the change from baseline to month 21 was used as response variable, treatment as explanatory variable and baseline value as a covariate. Factors such as age, gender, duration of diabetes at intervention, GADA titre, and HLA type were identified in advance as possible factors for additional exploratory analyses.

In all tests, the null hypothesis was that there is no difference between active treatment and placebo. For all tests two-sided hypotheses were used and the p-values presented together with 95% confidence intervals. As there is only one primary analysis, the p-values are not adjusted for multiplicity.

### Results Upon Completion of the 21-Month Visit

### Recruitment and Randomization

Of 118 patients screened, 42 girls and 28 boys were eligible. The screening took place over two weeks in January and February 2005. The first patient injection was in February 2005, and the last patient completed the 21-month visit in February 2007.

All but one patient received two doses of either GAD-alum or placebo (Figure A-21). One patient (girl, placebo) was withdrawn from the study due to confirmed infectious mononucleosis with icterus and received only one injection. Sixty-nine patients, 35 treated and 34 placebo, were included in the per protocol analysis. No “intention-to-treat” analysis was performed as only one patient (placebo) dropped out.

TABLE 6
Baseline demographic and clinical characteristics of treatment groups

| | GAD-alum | Placebo |
|---|---|---|
| Characteristic | (n = 35) | (n = 34) |
| Mean age ± SD, years | 13.8 ± 2.3 | 12.8 ± 1.9 |
| Mean duration of diabetes ± SD, months | 9.9 ± 5.3 | 8.8 ± 5.4 |
| Mean BMI ± SD, kg/m2 | 19.5 ± 2.4 | 20.5 ± 3.2 |
| Gender distribution, n (%) | | |
| Female | 23 (66) | 18 (53) |
| Male | 12 (34) | 16 (47) |
| HLA classification, n (%) | | |
| Very High Risk (VH) | 8 (23) | 9 (26) |
| High Risk (H) | 10 (29) | 7 (21) |
| Moderate Risk (M) | 9 (26) | 7 (21) |
| Neutral (N) | 4 (11) | 4 (12) |
| Low Risk (L) | 4 (11) | 7 (21) |
| Puberty stage at screening (Tanner Genital | | |
| Organs Stage), n (%) | | |
| Stage 1 | 4 (11) | 7 (20) |
| Stage 2-3 | 8 (23) | 10 (29) |
| Stage 4-5 | 23 (66) | 17 (50) |
| Mean insulin dose/kg | 0.66 ± 0.30 | 0.66 ± 0.28 |
| bodyweight ± SD, U/kg | | |
| Mean blood glucose prior to | 9.4 ± 4.0 | 8.8 ± 3.3 |
| MMTT ± SD, mmol/l | | |
| Mean HbA1c ± SD, % | 6.3 ± 1.3 | 6.2 ± 1.0 |
| Mean fasting C-peptide ± | 0.33 ± 0.19 | 0.35 ± 0.23 |
| SD, pmol/ml | | |
| Mean stimulated C-peptide | 0.78 ± 0.36 | 0.86 ± 0.54 |
| Maximum ± SD, pmol/ml | | |
| Mean stimulated C-peptide | 1.24 ± 0.57 | 1.41 ± 0.87 |
| AUC ± SD, pmol/ml * 2 hours | | |
| Median GADA titre, U/ml | ≧500 | ≧500 |
| GADA titre <500 U/ml, n (%) | 15 (43) | 11 (32) |
| GADA titre ≧500 U/ml, n (%) | 20 (57) | 23 (68) |

### Baseline Characteristics

Baseline data (baseline=day of first injection, prior to injection) shows that the two treatment groups were similar (Table 1). The distribution of HLA genotypes did not differ between the GAD-alum treated and the placebo group (Table 6).

### Safety

There were no treatment related serious adverse events in the study. An equal number, x in each group, of mild skin reactions (erythema, edema and tenderness) at the injection site in the GAD-alum and the placebo groups was observed. None required treatment or led to refusal of the second injection. Neurological assessment, based on the potential concern of inducing stiff person syndrome, indicated no difference between the study groups to date.

### Efficacy

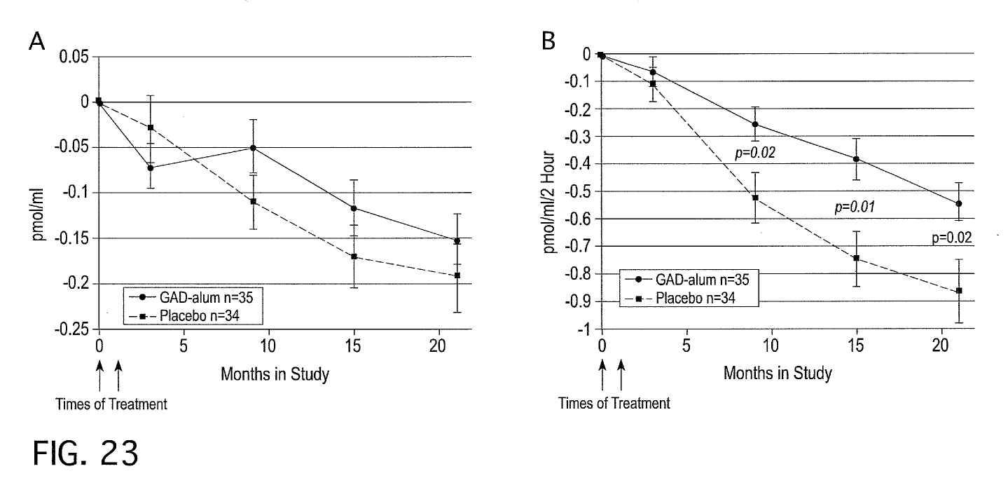

Both treatment groups showed a progressive decrease from baseline regarding both fasting and stimulated C-peptide secretion, indicating a gradual loss of beta cell function. There was no significant effect of treatment on fasting C-peptide (FIG. 18A and Table 7). However, over the 21 months, stimulated C-peptide secretion, as measured by AUC, decreased significantly less in the GAD-alum treated group than in the placebo group (p=0·01 for all time points throughout the study) (FIG. 18B and Table 7). Maximum stimulated C-peptide, being a part of AUC, also deteriorated significantly less in the GAD-alum treated group (p=0·03) (Table 7).

### Effect on Diabetes Status

Both treatment groups increased their insulin requirement and HbA1c level during the study (Table 7). HbA1c did not differ between the groups as it was used by physicians as a therapy target (Table 7). There was no significant difference regarding number of patients with GADA titre above 500 at 21 months (Table 8).

TABLE 7
Endpoints, absolute values at month 21 and mean change from baseline to month 21

| | Mean value at | | | | | |
|---|---|---|---|---|---|---|
| | month 21 ± SD | | Mean change from baseline ± SD | | | |
| | GAD-alum | Placebo | GAD-alum | Placebo | Treatment Effect* | p-value |
| Characteristic | (n = 35) | (n = 34) | (n = 35) | (n = 34) | (95% C.I.) | (ANCOVA) |
| Fasting C-peptide, pmol/ml | 0.18 ± 0.13 | 0.16 ± 0.20 | −0.15 ± 0.17 | −0.19 ± 0.23 | 0.03 (−0.04, 0.10) | 0.46 |
| Stimulated C-peptide Maximum, pmol/ml | 0.48 ± 0.42 | 0.36 ± 0.39 | −0.33 ± 0.25 | −0.50 ± 0.41 | 0.15 (0.01, 0.30) | 0.03 |
| Stimulated C-peptide AUC, pmol/ml * 2 hour | 0.73 ± 0.59 | 0.55 ± 0.60 | −0.54 ± 0.40 | −0.86 ± 0.67 | 0.26 (0.04, 0.48) | 0.02 |
| Insulin dose/kg bodyweight, U/kg | 0.88 ± 0.32 | 0.93 ± 0.29 | 0.22 ± 0.26 | 0.27 ± 0.35 | −0.05 (−0.18, 0.08) | 0.45 |
| HbA1c, % | 7.1 ± 1.5 | 6.9 ± 1.3 | 0.8 ± 1.5 | 0.6 ± 1.4 | 0.2 (−0.5, 0.8) | 0.56 |

*Treatment effect estimated using least square means methodology with baseline value as covariate.

TABLE 8

| | | | p-value |
|---|---|---|---|
| | GAD-alum | Placebo | (Fisher's |
| | (n = 35) | (n = 34) | Exact Test) |
| Median GADA titre, U/ml | ≧500 | ≧500 | — |
| GADA titre <500 U/ml, n (%) | 10 (29) | 17 (50) | 0.09 |
| GADA titre ≧500 U/ml, n (%) | 25 (71) | 17 (50) | |

The statistically significant effect of treatment on change in stimulated C-peptide remained after adjusting for differences in duration of diabetes, age, gender, and baseline GADA levels (data not shown).

The subgroups specified in the protocol regarding duration of diabetes, age, gender, HLA and baseline GADA levels were also investigated for interaction effects (data not shown). Of these, only duration of diabetes had a significant influence on the efficacy of treatment (p=0·04).

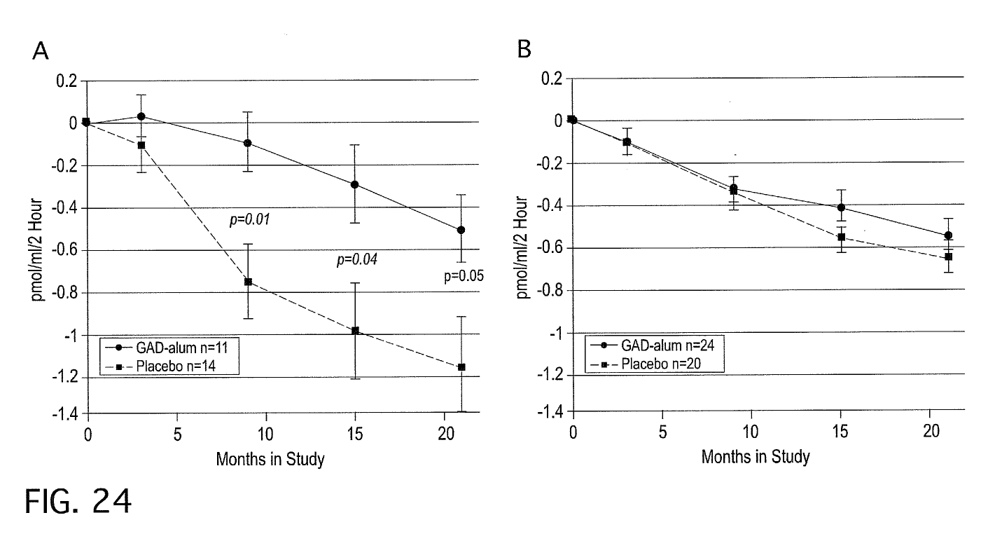

The preservation of residual insulin secretion was more pronounced among patients treated with GAD-alum soon after T1D onset. In patients treated within 6 months of diagnosis stimulated C-peptide secretion, as measured by AUC throughout the study period, decreased significantly less in the GAD-alum treated group than in the placebo group (p=0·02), while no such difference could be observed in patients with a duration of diabetes of 6 months or more (FIG. 19).

### Discussion

Despite the lack of effect on fasting C-peptide, which was chosen as the primary endpoint based on the previous study in LADA patients, there was a highly similar to observations from other immune intervention studies^{16, 17} no significant beneficial effect of GAD-alum treatment in C-peptide responses²⁴. Fasting C-peptide levels in recent onset T1D patients may remain near normal when fasting glucose is kept near normal²⁹ while stimulated C-peptide levels usually decline over time as a consequence of continuous beta cell destruction. As a result early in the course of the disease, stimulated C-peptide has tended to be the endpoint for assessing preservation of beta cell function, since it is correlated with both improved glycemic control and less micro-vascular complications.^{16,17, 31}

The foregoing results demonstrate that two injections of 20 μg GAD-alum significantly improves preservation of residual stimulated insulin secretion in patients with recent onset T1D during a follow-up time of similar length as in other intervention studies.^{14,16,17} In the newly-diagnosed patients, the effect size and duration is similar with GAD-alum treatment as with antiCD3^{16,17}, but differs in so far as GAD-alum treatment has been without adverse events. Residual insulin secretion affects important clinical outcome parameters.⁵ Although not limited to the theory of the invention, this may be explained by improved overall metabolic control, less blood glucose fluctuations and possibly, by greater C-peptide exposure, which itself may have biologic effects.²⁹

Both treatment groups increased both their insulin requirement and HbA1c level during the study and, as hoped, the aggressive treatment to HbA1c target did not result in any significant difference in HbA1c between the groups. Nor was there any significant difference in GADA levels between the two groups. The effect of our immunomodulatory vaccine formulation using GAD65 on other autoantibodies as well as on cell-mediated immunity will be subject to further analysis.

Minimal and insignificant differences in the demographics of the drug treated patients and the placebo patients are unlikely to be relevant. While the drug treated patients were slightly older, the placebo patients had a somewhat shorter disease duration and, on average, higher residual insulin secretion at baseline. It was important to carefully evaluate the possible role of HLA as it has been shown that GADA in T1D patients are associated with HLA-DQA1*0501-B1*02.²⁷ However, the distribution of HLA genotypes were comparable and could not explain the highly differences in C-peptide responses between the GAD-alum and placebo groups. If anything, the GAD-alum patients had fewer neutral and low risk alleles (n=22) compared to the placebo patients (n=36).⁷ Further studies in larger number of patients will be needed to evaluate the possible importance of HLA genotypes in response to GAD-alum.

As a previous study had suggested effect of GAD-alum vaccination in LADA patients with slowly progressive autoimmune diabetes, we chose to include not only very recently diagnosed T1D patients, but those with duration up to 18 months. However, the protective effect of the GAD-alum treatment on stimulated C-peptide tended to be especially pronounced in patients treated <6 months after diagnosis.

The mechanism of how GAD-alum treatment altered disease progression in TlD patients is not clear. The intense insulin treatment and the ability to achieve the target HbA1c in all patients, as well as the minimal differences in HbA1c and insulin dose between groups, should exclude the significant differences in preserved beta cell function being attributed to more intense insulin treatment or better metabolic control in the GAD-alum group. Animal studies in NOD mice have shown that GAD65 can induce potent regulatory response in mice with established autoimmunity and after the onset of T1D.^{22,23} It is of interest in this regard that the previous dose-finding study revealed that GAD-alum induced an increase in the (CD4+CD25+)/(CD4+CD25−) cell ratio at 24 weeks suggesting a possible effect on regulatory (CD4+CD25+) T cells.²⁴

In conclusion, treatment with alum-formulated GAD65 had a protective effect on residual beta cell function 21 months after intervention with infrequent and minor adverse side-effects. The demonstrated efficacy of GAD-alum, in line with or better than other developing treatments reported thus far, gives hope that autoantigen-specific immunomodulation, alone or used in combination therapy, may eventually prevent T1D. It is promising that the protective effect on beta cell function in this study occurred with the use of a drug which is very easy to administer and well tolerated.

### Study Design

At eight pediatric clinics in Sweden, 10-18 year old T1D patients who had presented with disease within the previous 18 months were screened for presence of GAD65 autoantibodies (GADA) and fasting C-peptide levels above 0.1 pmol/ml. A total of 70 patients were eligible and randomized to a double-blind treatment of either 20 μg of recombinant human GAD65 formulated in alum (Diamyd®, Diamyd Medical, Stockholm, Sweden; 35 patients) or placebo (the same formulation without rhGAD65; 35 patients).

All patients were treated with Multiple Insulin Therapy and both the patients and their parents or guardians provided informed consent. The trial objective was to evaluate the safety as well as the efficacy of treatment compared to placebo in preserving residual insulin secretion. The primary efficacy endpoint was change in fasting C-peptide level from baseline to month 15. The secondary efficacy endpoints were changes from baseline in stimulated C-peptide levels and HbA1c.

Each patient received a subcutaneous primary injection of either GAD65 or placebo on day 1 followed by a boost one month later. Patients remained in the clinic to be observed for three hours after injection.

On day 1 and at months 3, 9 and 15, a mixed meal tolerance test (MMTT) was performed in accordance with the European study on estimation of beta cell function²⁵ which includes ingestion of 6 ml Sustacal®/kg body weight (Sustacal®, Mead Johnson, Evansville, Ind., USA). Blood samples for C-peptide analysis were collected before, 30, 60, 90 and 120 minutes after start of the MMTT. Safety evaluations including neurological assessments, clinical examination, hematology, biochemistry and impact of treatment on diabetes status were repeatedly assessed throughout the study.

After completion of the main study period (15 months), the treatment code was opened, and data analyzed including C-peptide levels (fasting, max, area under the curve [AUC]), HbA1c, blood glucose, insulin requirement (units per kg body weight and 24 hours) and GADA titre.

With only the statistician, the SAS programmer and the sponsor being informed of unblinded data, the study continued in a partly blinded fashion for an extended period of 15 months.

### Laboratory Tests

Laboratory analyses were performed at Linkoping University, Sweden. C-peptide levels were measured in serum samples with a Time-resolved fluoroimmunoassay (AutoDELFIA™ C-peptide kit, Wallac, Turku, Finland). Results were validated with inclusion of a C-peptide control module containing a high, a medium and a low-level control in each assay (commercially available from Immulite, DPC, UK). A 1224 MultiCalc® program (commercially available from Wallac) was used for automatic measurement and result calculation and measurements were expressed in pmol/ml.

Serum GADA titres were determined in duplicate using a radiobinding assay employing ³⁵S-labelled recombinant human GAD65 produced by in vitro transcription/translation (pEx9 vector kindly supplied by Prof. Ake Lernmark, University of Washington, Seattle, Wash., USA). Sepharose protein A was used to separate free from antibody-bound labeled GAD65. GADA levels are presented with a maximum of 500 U/ml as it was decided to determine maximal titres at the end of the study. HLA-DQ A1* and B1* alleles were determined by PCR amplification of exon 2 sequences and hybridization with allele-specific probes detected by time-resolved fluorescence as described.²⁶ As detailed in a population-based Swedish case-control study²⁷, the patients were then divided into very high risk, high risk, moderate risk, neutral and low risk subjects.

### Statistical Analysis

Results from a previous study in LADA patients²⁴ suggested that 35 patients in each treatment group would provide a power of 80-90% for assessing differences in C-peptide levels, with a significance level of 5%, assuming a mean difference of 0.12 pmol/ml and a standard deviation of 0.15 in fasting C-peptide levels. Data management and the statistical analysis were performed by Trial Form Support AB, Lund, Sweden. An analysis of covariance (ANCOVA) model was used in which the change from baseline to month 15 was used as response variable, treatment as explanatory variable and baseline value as a covariate. Factors such as age, gender, duration of diabetes at intervention, GADA titre, and HLA type were identified in advance as possible factors for additional exploratory analyses.

In all tests the null hypothesis was that there is no difference between active treatment and placebo. For all tests two-sided hypotheses were used and the p-values presented together with 95% confidence intervals. As there is only one primary analysis, the p-values are not adjusted for multiplicity.

### Recruitment and Randomization

Of 118 patients screened, 42 girls and 28 boys were eligible. The screening took place during two weeks in January and February 2005. The first patient injection was in February 2005 and the last patient completed the 15-month visit in July 2006.

All but one patient received two doses of either GAD65 or placebo (Figure D). One patient (girl, placebo) was withdrawn from the study due to mononucleosis with icterus and received only one injection. Sixty-nine patients, 35 Diamyd® treated and 34 placebo, were included in the per protocol analysis. No “intention-to-treat” analysis was performed as only one patient (placebo) dropped out.

TABLE 1
Baseline demographic and clinical characteristics of treatment groups

| | Diamyd ® | Placebo |
|---|---|---|
| Characteristic | (n = 35) | (n = 34) |
| Mean age ± SD, years | 13.8 ± 2.3 | 12.8 ± 1.9 |
| Mean duration of diabetes ± SD, | 9.9 ± 5.3 | 8.8 ± 5.4 |
| months | | |
| Mean BMI ± SD, kg/m2 | 19.5 ± 2.4 | 20.5 ± 3.2 |
| Gender distribution, n (%) | | |
| Female | 23 (66) | 18 (53) |
| Male | 12 (34) | 16 (47) |
| HLA classification, n (%) | | |
| Very High Risk (VH) | 8 (23) | 9 (26) |
| High Risk (H) | 10 (29) | 7 (21) |
| Moderate Risk (M) | 9 (26) | 7 (21) |
| Neutral (N) | 4 (11) | 4 (12) |
| Puberty stage at screening (Tanner | 4 (11) | 7 (20) |
| Genital Organs Stage), n (%) | | |
| Stage 1 | 8 (23) | 10 (29) |
| Mean insulin dose/kg bodyweight ± SD, | 0.66 ± 0.30 | 0.66 ± 0.28 |
| U/kg | | |
| Mean blood glucose prior to MMTT ± SD, | 9.4 ± 4.0 | 8.8 ± 3.3 |
| mmol/l | | |
| Mean HbA1c ± SD, % | 6.3 ± 1.3 | 6.2 ± 1.0 |
| Mean fasting C-peptide ± SD, pmol/ml | 0.33 ± 0.19 | 0.35 ± 0.23 |
| Mean stimulated C-peptide Maximum ± SD, | 0.78 ± 0.36 | 0.86 ± 0.54 |
| pmol/ml | | |
| Mean stimulated C-peptide AUC ± SD, | 1.24 ± 0.57 | 1.41 ± 0.87 |
| pmol/ml * 2 hours | | |
| Median GADA titre, U/ml | ≧500 | ≧500 |
| GADA titre <500 U/ml, n (%) | 15 (43) | 11 (32) |
| GADA titre ≧500 U/ml, n (%) | 20 (57) | 23 (68) |

### Baseline Characteristics

Baseline data (baseline=day of first injection, prior to injection) shows that the two treatment groups were similar in most aspects (Table 1). The distribution of HLA genotypes did not differ between the Diamyd® and the placebo group (Table 1).

### Safety

There were no treatment-related serious adverse events in the study. An equal number of mild skin reactions (erythema, edema and tenderness) were observed at the injection site in both the Diamyd® and the placebo groups. None required treatment or led to refusal of the second injection. Neurological assessment, based on the potential concern of inducing Stiff Person Syndrome, indicated no difference between the study groups.

TABLE 2
Endpoints, absolute values and mean change from baseline to month 15
(Absolute C-peptide values may be added into this Table)

| | | | Treatment | |
|---|---|---|---|---|
| Mean change in characteristic ± | Diamyd ® | Placebo | Effect* | p-value |
| SD | (n = 35) | (n = 34) | (95% C.I.) | (ANCOVA) |
| ΔFasting C-peptide, pmol/ml | −0.12 ± 0.18 | −0.17 ± 0.20 | 0.04 (−0.04, 0.12) | 0.28 |
| ΔStimulated C-peptide Maximum, | −0.24 ± 0.26 | −0.42 ± 0.40 | 0.16 (0.01, 0.31) | 0.04 |
| pmol/ml | | | | |
| ΔStimulated C-peptide AUC, | −0.38 ± 0.46 | −0.75 ± 0.61 | 0.30 (0.07, 0.54) | 0.01 |
| pmol/ml * 2 hour | | | | |
| ΔInsulin dose/kg bodyweight, U/kg | 0.15 ± 0.22 | 0.22 ± 0.29 | −0.08 (−0.19, 0.04) | 0.19 |
| ΔBlood glucose prior to MMTT, | 0.1 ± 5.8 | 1.3 ± 5.8 | −0.6 (−2.8, 1.6) | 0.57 |
| mmol/l | | | | |
| ΔHbA1c, % | 0.3 ± 1.3 | 0.5 ± 1.5 | −0.2 (−0.7, 0.4) | 0.57 |

*Treatment effect estimated using least square means methodology with baseline value as covariate.

TABLE 3
GADA titre at month 15

| | Diamyd ® | Placebo | p-value |
|---|---|---|---|
| | (n = 35) | (n = 34) | (Fisher's Exact |
| Median GADA titre, U/ml | ≧500 | ≧500 | — |
| GADA titre <500 U/ml, n (%) | 7 (20) | 14 (41) | 0.07 |
| GADA titre ≧500 U/ml, n (%) | 28 (80) | 20 (59) | |

### Exploratory Analyses and Interaction

Of the patients who had a maximum stimulated C-peptide above 0.2 pmol/ml at baseline, only 19% of the Diamyd®-treated patients fell below that limit by month 15 compared to 42% of the placebo-treated patients (Table 4).

TABLE 4
Patients with a maximum stimulated C-peptide >0.2 pmol/ml

| | Diamyd ® | Placebo |
|---|---|---|
| Visit | (n = 35) | (n = 34) |
| Baseline, n (% of baseline value) | 32 (100) | 33 (100) |
| Month 15, n (% of baseline value) | 26 (81) | 19 (58) |
| Change, n (% of baseline value) | −6 (−19) | −14 (−42) |

Covariate analyses of the changes in fasting and stimulated C-peptide from baseline to month 15 for 69 patients were performed using baseline C-peptide and treatment as covariates together with either duration of diabetes, age, gender, or baseline GADA levels. These analyses revealed that only baseline C-peptide levels and treatment with Diamyd® had a statistically significant effect on residual insulin secretion during follow up (data not included).

The subgroups specified in the protocol regarding duration of diabetes, age, gender, baseline GADA levels and HLA classification were investigated for effects on the efficacy of GAD65. For this purpose the efficacy was defined as reduction of the deterioration in AUC from baseline until month 15 in the Diamyd® group compared to placebo. No formal statistical analysis was conducted due to small sample sizes and potential issues of multiple comparisons. (Table 5)

In all tests the null hypothesis was that there is no difference between active treatment and placebo The preservation of residual insulin secretion was more pronounced among patients treated shortly after T1D onset (Figure E, Table 5).

TABLE 5
Mean change ± SD in stimulated C-peptide AUC from
baseline to Month 15 for sub groups

| Sub group | Diamyd ® | | | Placebo | | |
|---|---|---|---|---|---|---|
| (classification | (pmol/ml * 2 hour) | | | (pmol/ml * 2 hour) | | |
| at baseline) | n | Baseline | Change | n | Baseline | Change |
| Duration | | | | | | |
| 0-3 months | 4 | 1.52 ± 0.34 | +0.10± | 7 | 1.63 ± 0.55 | −0.89± |
| 3-6 months | 7 | 1.49 ± 0.54 | −0.53± | 7 | 1.72 ± 1.24 | −1.08± |
| 6-12 months | 7 | 0.90 ± 0.49 | −0.46± | 9 | 1.48 ± 0.88 | −0.71± |
| 12-18 months | 17 | 1.21 ± 0.61 | −0.40± | 11 | 1.03 ± 0.70 | −0.44± |
| Age | | | | | | |
| 10-12 years | 11 | 1.21 ± 0.56 | −0.50± | 18 | 1.30 ± 0.71 | −0.69± |
| 13-15 years | 15 | 1.17 ± 0.58 | −0.36± | 13 | 1.28 ± 0.72 | −0.72± |
| 16-18 years | 9 | 1.38 ± 0.61 | −0.27± | 3 | 2.68 ± 1.52 | −1.15± |
| Gender | | | | | | |
| Females | 23 | 1.32 ± 0.66 | −0.34± | 18 | 1.56 ± 0.95 | −0.91± |
| Males | 12 | 1.08 ± 0.31 | −0.45± | 16 | 1.25 ± 0.76 | −0.57± |
| GADA titre | | | | | | |
| <500 U/ml | 15 | 1.25 ± 0.50 | −0.52± | 11 | 1.18 ± 1.11 | −0.77± |
| ≧500 U/ml | 20 | 1.23 ± 0.63 | −0.27± | 23 | 1.53 ± 0.72 | −0.73± |
| HLA | | | | | | |
| classification | | | | | | |
| Very High Risk | 8 | 1.02 ± 0.74 | −0.33± | 9 | 1.48 ± 0.77 | −0.85± |
| High Risk | 10 | 1.09 ± 0.51 | −0.26± | 7 | 1.39 ± 0.88 | −0.98± |
| Moderate Risk | 9 | 1.56 ± 0.54 | −0.63± | 7 | 1.54 ± 1.22 | −0.88± |
| Neutral Risk | 4 | 1.38 ± 0.34 | −0.48± | 4 | 0.89 ± 0.63 | −0.16± |
| Low Risk | 4 | 1.19 ± 0.43 | −0.10± | 7 | 1.53 ± 0.81 | −0.53± |

### Discussion

Current opinion holds that stimulated C-peptide is a preferred endpoint for assessing preservation of beta cell function in type 1 diabetes patients and this is correlated with improved glycemic control and fewer micro-vascular complications.³¹

The results demonstrate that two injections of 20 μg GAD65 significantly improves preservation of residual insulin secretion in patients with recent onset T1D during a follow-up time of similar length as in other intervention studies (14,16,17). Furthermore, a smaller proportion of patients in the GAD65 than in the placebo group lost their capacity to secrete more than 0.2 pmol/ml of C-peptide in response to a meal. In the Diabetes Control and Complications Trial (DCCT), patients with less than 0.2 pmol/ml in stimulated C-peptide had a higher risk for retinopathy and severe hypoglycemia than patients above that threshold.^{5,28} This may be explained by improved overall metabolic control, less blood glucose fluctuations and possibly by greater C-peptide exposure, which itself may have biological effects.²⁹ To our knowledge there are few clinical trials with an immunomodulatory approach that have achieved such a reduced loss of residual insulin secretion as in the present study.

As a previous study had suggested an effect of GAD65 vaccination in LADA patients with slowly progressive autoimmune diabetes, we chose to include not only newly-diagnosed T1D patients but also those with a duration of up to 18 months prior to first treatment. However, the protective effect of Diamyd® on stimulated C-peptide tended to be especially pronounced in patients treated shortly after diagnosis. Our design makes subgroup analyses difficult, but if confirmed, patients with short disease duration and good residual insulin secretion might improve their function enough to go into complete remission, whereby the ongoing autoimmune attack will be abated by the treatment.

The mechanism of how Diamyd® treatment altered disease progression in T1D patients is not yet clear. The same type of insulin treatment in all patients, and the small difference in HbA1c should exclude that the preserved beta cell function is the consequence of more intense insulin treatment or better metabolic control in the GAD65 group. Numerous animal studies in NOD mice have shown that GAD65 can induce potent immune regulatory response in mice with established autoimmunity and after the onset of T1D.^{22,23} It is of interest in this regard that the prior dose-finding study revealed that Diamyd® induced an increase in the (CD4+CD25+)/(CD4+CD25−) cell ratio at 24 weeks, suggesting a possible effect on regulatory (CD4+CD25+) T cells.²⁴

In conclusion, the treatment with alum-formulated GAD65 had a protective effect on residual beta cell function 15 months following intervention. The demonstrated efficacy of GAD65, in concordance with or better than other developing treatments reported so far, gives hope that autoantigen-specific immunomodulation, alone or used in combination therapy, may eventually prevent T1D. It is promising that the protective effect on beta cell function in this study occurred with the use of a drug which is very easy to administer and well tolerated with mild and few adverse events.

### Efficacy

The preservation of residual insulin secretion was more pronounced among patients treated with GAD-alum soon after T1D onset. In patients treated within 6 months of diagnosis stimulated C-peptide secretion, as measured by AUC throughout the study period, decreased significantly less in the GAD-alum treated group than in the placebo group (p=0·02), while no such difference could be observed in patients with a duration of diabetes of 6 months or more (FIG. 19). In the group of patients that had been diagnosed with the disease within six months a deterioration of stimulated C-peptide is seen as a function of time and subgroup analysis indicates that the drug is suitable to be used three times within an approximate three month period. Increased deterioration of stimulated C-peptide at around 6-15 months after treatment initiation indicates that the study drug is suitable to be used four times within a 12 month period for long-term preservation of beta cell function.

In the newly-diagnosed patients the effect size and duration is similar with GAD-alum treatment as with anti-T cell therapies^{16,17}, but differs in so far as GAD-alum treatment has been without adverse events.

### REFERENCES

1. Atkinson M A, Eisenbarth G S. Type 1 diabetes: new perspectives on disease pathogenesis and treatment. Lancet 2001; 358:221-9.
2. The effect of intensive treatment of diabetes on the development and progression of long-term complications in insulin-dependent diabetes mellitus. The Diabetes Control and Complications Trial Research Group. N Engl J Med 1993; 329:977-86.
3. Bojestig M, Arnqvist H J, Hermansson G, Karlberg B E, Ludvigsson J. Declining incidence of nephropathy in insulin-dependent diabetes mellitus. N Engl J Med 1994; 330: 15-8.
4. Nordfeldt S, Ludvigsson J. Adverse events in intensively treated children and adolescents with type 1 diabetes. Acta Paediatr 1999; 88:1184-93.
5. Steffes M W, Sibley S, Jackson M, Thomas W. Beta-cell function and the development of diabetes-related complications in the Diabetes Control and Complications Trial. Diabetes Care 2003; 26:832-6.
6. Ludvigsson J, Heding L, Lieden G, Marner B, Lernmark A. Plasmapheresis in the initial treatment of insulin-dependent diabetes mellitus in children. Br Med J (Clin Res Ed) 1983; 286:176-8.
7. Dupre J, Stiller C R, Gent M, et al. Clinical Trials of Cylosporin in IDDM. Diabetes Care 1988; suppl 1:37-44.
8. Eisenbarth G S, Srikanta S, Jackson R, et al. Anti-thymocyte globulin and prednisone immunotherapy of recent onset type 1 diabetes mellitus. Diabetes Research 1985; 2:271-6.
9. Chase H P, Butler-Simon N, Garg S, McDuffie M, Hoops S L, O'Brien D. A trial of nicotinamide in newly diagnosed patients with type 1 (insulin-dependent) diabetes mellitus. Diabetologia 1990; 33:444-6.
10. Pozzilli P, Visalli N, Signore A, et al. Double blind trial of nicotinamide in recent-onset IDDM (the IMDIAB III study). Diabetologia 1995; 38:848-52.
11. Coutant R, Landais P, Rosilio M, et al. Low dose linomide in Type I juvenile diabetes of recent onset: a randomised placebo-controlled double blind trial. Diabetologia 1998; 41:1040-6.
12. Ludvigsson J, Samuelsson U, Johansson C, Stenhammar L. Treatment with antioxidants at onset of type 1 diabetes in children: a randomized, double-blind placebo-controlled study. Diabetes Metab Res Rev 2001; 17:131-6.
13. Ludvigsson J, Samuelsson U, Ernerudh J, Johansson C, Stenhammar L, Berlin G. Photopheresis at onset of type 1 diabetes: a randomised, double blind, placebo controlled trial. Arch Dis Child 2001; 85:149-54.
14. Raz I, Elias D, Avron A, Tamir M, Metzger M, Cohen I R. Beta-cell function in new-onset type 1 diabetes and immunomodulation with a heat-shock protein peptide (DiaPep277): a randomised, double-blind, phase II trial. Lancet 2001; 358:1749-53.
15. Lazar L, Ofan R, Weintrob N, et al. Heat-shock protein peptide DiaPep277 treatment in children with newly diagnosed type 1 diabetes: a randomised, double-blind phase II study. Diabetes Metab Res Rev 2006; (Epub ahead of print).
16. Herold K C, Gitelman S E, Masharani U, et al. A single course of anti-CD3 monoclonal antibody hOKT3gamma1(Ala-Ala) results in improvement in C-peptide responses and clinical parameters for at least 2 years after onset of type 1 diabetes. Diabetes 2005; 54:1763-9.
17. Keymeulen B, Vandemeuleroucke E, Ziegler A G, et al. Insulin needs after CD3-antibody therapy in new-onset type 1 diabetes. N Engl J Med 2005; 352:2598-608.
18. Harrisson L C. The prospect of vaccination to prevent type 1 diabetes. Hum Vaccin 2005; 1:143-50.
19. Jasinski J M, Eisenbarth G S. Insulin as a primary autoantigen for type 1A diabetes. Clin Dev Immunol 2005; 12:181-6.
20. Baekkeskov S, Nielsen J H, Marner H, Bilde T, Ludvigsson J, Lernmark A. Autoantibodies in newly diagnosed diabetic children immunoprecipitate human pancreatic islet cell proteins. Nature 1982; 298:167-9.
21. Lernmark A, Agardh C D. Immunomodulation with human recombinant autoantigens. Trends Immunol 2005; 26:608-12.
22. Tian J, Clare-Salzler M, Herschenfeld A, et al. Modulating autoimmune responses to GAD inhibits disease progression and prolongs islet graft survival in diabetes-prone mice. Nature Medicine 1996; 2:1348-135.
23. Tisch R, Liblau R S, Yang X D, Liblau P, McDevitt H O. Induction of GAD65-specific regulatory T cells inhibits ongoing autoimmune diabetes in non-obese diabetic mice. Diabetes 1998; 47:894-9.
24. Agardh C D, Cilio C M, Lethagen A, et al. Clinical evidence for the safety of GAD65 immunomodulation in adult-onset autoimmune diabetes. J Diabetes Complications 2005; 19:238-46.
25. Mandrup-Poulsen T R, Battelino T, Haastert B, et al. The European C-peptide trial CPT: comparison of liquid mixed meal and glucagons stimulation assays for the analyses of residual β-cell function in type 1 diabetes. Diabetologia 2006; 49:375.
26. Larsson H E, Lynch K, Lernmark B, et al. DiPiS Study Group. Diabetes-associated HLA genotypes affect birthweight in the general population. Diabetologia 2005; 48:1484-91.
27. Graham J, Hagopian W A, Kockum I, et al. Diabetes Incidence in Sweden Study Group; Swedish Childhood Diabetes Study Group. Genetic effects on age-dependent onset and islet cell autoantibody markers in type 1 diabetes. Diabetes 2002; 51:1346-55.
28. The Diabetes Control and Complications Trial Research Group. Effect of intensive therapy on residual β-Cell function in patients with type 1 diabetes in the diabetes control and complications trial. A randomized controlled trial. Ann Intern Med. 1998; 128:517-23.
29. Wahren J, Ekberg K, Johansson J, et al. Role of C-peptide in human physiology. Am J Physiol Endocrinol Metab 2000; 278:E759-60.
30. Daneman D, Clarson C. Residual beta-cell function in children with type 1 diabetes: measurement and impact on glycemic control. Clin Invest Med 1987; 10:484-7.
31. Steele C, Hagopian W A, Gitelman S, et al. Insulin secretion in type 1 diabetes. Diabetes 2004; 53:426-33.

All publications and patents mentioned herein are hereby incorporated by reference to the same extent as if each individual publication or patent was specifically and individually indicated to be incorporated by reference. Many variations of the present invention within the scope of the appended claims will be apparent to those skilled in the art once the principles described herein are understood.

What is claimed is:

1. A method for treating an autoimmune disease comprising the steps of (1) identifying a patient determined to have type 1 diabetes having progressed for not longer than 6 months, and (2) initiating treatment by administering a medicament comprising at least one autoantigen within said 6 month period.

2. A method according to claim 1 wherein said treatment initiation occurs before said type 1 diabetes has progressed for no longer than 3 months.

3. A method according to claim 1 wherein said medicament is administered at least three times within 6 months from treatment initiation.

4. A method according to claim 1 wherein said medicament is administered at least three times within 3 months from treatment initiation.

5. A method according to claim 1 wherein said medicament is administered at least four times within 12 months from treatment initiation.

6. A method according to claim 1 wherein said medicament is administered at least four times within 10 months from treatment initiation.

7. A method according to claim 1 wherein said medicament is administered at least two times within 40 days from treatment initiation.

8. A method for treating an autoimmune disease comprising the steps of (1) identifying a patient determined to have an autoimmune disease having progressed no longer than that fasting C-peptide levels are greater than 0.1 pmol/ml, and (2) initiating treatment by administering a medicament comprising at least one autoantigen before said fasting C-peptide level diminishes below 0.1 pmol/ml.

9. A method according to claim 1 wherein said at least one autoantigen being one from the constructs selected from the group consisting of GAD65, GAD67, Pro-Insulin, Basic Myelin Protein, MOG, Collagen Type II, ICA512 (IA2), ICA512B (IA2B), insulin, insulin B-chain, Hsp60, Hsp65, P277, ICA69, Glima38, SOX13, Imogen 38, Sulfatide, 21-Ohase, TPO, allergens, transplant antigens, cancer antigens, or parts, peptides or altered peptide ligands thereof.

10. A method according to claim 1 wherein said medicament is characterized by having the constructs formulated in a Th2-driving adjuvant.

11. A method according to claim 10 wherein said Th2 driving adjuvant is alum.

12. The method according to claim 1 wherein said medicament is administered by at least one of the routes selected from the group consisting of oral, nasal, inhaled, intramuscular, subcutaneous, intravenous, colorectal, transdermal, or by use of an implant or pump, use of DNA and RNA guns, or viral vectors.

13. The method according to claim 1 wherein the amount of at least one of the autoantigens is between about 10 and about 150 micrograms per treatment occasion.
